(19) United States
(12) Patent Application Publication
(10) Pub. No.: US 2018/0355099 A1
(43) Pub. Date: Dec. 13, 2018

(54) Fluorinated Benzoxadiazole-Based Donor-Acceptor Polymers for Electronic and Photonic Applications

(71) Applicant: The Hong Kong University of Science and Technology, Hong Kong (CN)

(72) Inventors: He YAN, Hong Kong (CN); Jingbo ZHAO, Henan (CN)

(21) Appl. No.: 15/781,863

(22) PCT Filed: Dec. 9, 2016
(86) PCT No.: PCT/CN2016/109131

§ 371 Date: Jun. 6, 2018

Related U.S. Application Data
(60) Provisional application No. 62/386,679, filed on Dec. 9, 2015.

(51) Int. Cl. C08G 61/12; H01L 51/00; H01L 51/42; H01L 51/05; C07D 271/12

(52) U.S. Cl. CPC C08G 61/126; H01L 51/0043; H01L 51/0036; C08K 3/045; H01L 51/0508; H01L 51/0004; C07D 271/12; H01L 51/4253

(57) ABSTRACT

A polymer comprising a repeating unit, which may further comprise one or more repeating units. The present subject matter also relates to a formulation comprising the polymer, and a fullerene, second polymer, or small molecule. The present subject matter further relates to an organic electronic (OE) device comprising a coating or printing ink containing the formulation, where the OE device may be an organic field effect transistor (OFET) device or an organic photovoltaic (OPV) device. The present subject matter also relates to synthesis of monomers, polymers, and the compounds produced therein.

### RELATED APPLICATIONS

The present patent application claims priority to provisional U.S. Patent Application No. 62/386,679 filed Dec. 9, 2015, which was filed by the inventors hereof and is incorporated by reference herein in its entirety.

### TECHNICAL FIELD

The present subject matter relates to novel donor-acceptor conjugated polymers, methods for their preparation and intermediates used therein, the use of formulations containing such polymers as semiconductors in organic electronic (OE) devices, especially in organic photovoltaic (OPV) and organic field-effect transistor (OFET) devices, and to OE and OPV devices made from these formulations.

### BACKGROUND

In recent years there has been growing interest in the use of organic semiconductors, including conjugated polymers, for various electronic applications.

One particular area of importance is the OPV field, where organic semiconductors (OSCs) allow devices to be manufactured by solution-processing techniques, such as spin casting and printing. Solution processing can be carried out cheaper and on a larger scale compared to the evaporative techniques used to make inorganic thin film devices.

The polymers commonly used in OSCs consist of electron donating (donor or D) and electron accepting (acceptor or A) co-monomer units. It is convenient to use such a D-A alternating copolymer strategy to obtain polymers with low optical bandgaps, as the HOMO level of the polymer is mostly located on the donor unit and the LUMO level mostly on the acceptor unit.

The commonly accepted model developed by Brabec, etc. indicates that an elaborately designed HOMO and LUMO energy level is a basic requirement for high-performance polymer solar cells because open-circuit voltage (V_{oc}) of polymer solar cells is determined by the difference between the HOMO level of the polymer and the LUMO level of the fullerene derivative. The LUMO energy level is relatively more important because LUMO offset between polymer and fullerene should be small enough to minimize V_{oc} loss. By modifying the acceptor unit with electron-donating or withdrawing groups, the LUMO level of the D-A polymer can be effectively tuned, while the same can be done to tune the HOMO level by modifying the donor unit.

To achieve higher V_{OC} and reduce energy loss, it is important to explore new building blocks to construct novel conjugated polymers. In several previous reports, it was shown that replacing the benzothiadiazole (BT) unit by its analogue benzoxadiazole (BX) could lead to a higher V_{OC} for the OSC devices while maintaining an almost identical optical bandgap. However, the polymers based on the BX building block generally yielded inferior OSC performance compared with their BT analogues. On the other hand, an important derivative of the BT unit is difluorobenzothiadiazole (ffBT) that has been widely explored for applications in OSCs, including the state-of-the-art single junction and tandem OSCs, ITO-free flexible OSCs, additive/annealing-free OSCs and so on. The success of the ffBT-based polymers can be attributed to their high polymer crystallinity and thus hole mobility, which lead to several cases of thick-film OSCs with high fill factors and efficiencies. The success of the ffBT unit may inspire one to develop a similar fluorinated building block based on BX, which could potentially combine the advantages of high polymer crystallinity/mobility and high V_{OC} without changing the bandgap. However, the synthesis of the difluorobenzoxadiazole (ffBX) unit is challenging and there has been no report of the ffBX based conjugated polymers.

### SUMMARY

In an embodiment, the present subject matter is directed to a polymer comprising one or more repeating units of formula I:

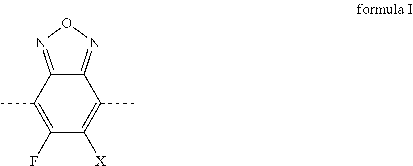

wherein X is H or F.

In an embodiment, the present subject matter is directed to a process of preparing a polymer or organic compound comprising polymerizing an intermediate with formula VIII:

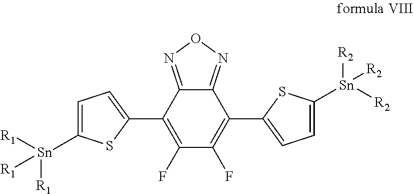

wherein R₁ and R₂, at each occurrence, independently can be a C₁₋₁₀ alkyl group.

In an embodiment, the present subject matter is directed to a process of preparing a polymer or organic compound comprising polymerizing an intermediate with formula IX:

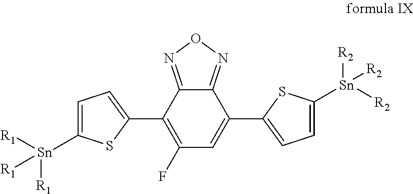

wherein R₁ and R₂, at each occurrence, independently can be a C₁₋₁₀ alkyl group.

In an embodiment, the present subject matter is directed to a formulation comprising the polymer of the present subject matter, and a fullerene, a second polymer, or a small molecule.

In an embodiment, the present subject matter is directed to an organic electronic (OE) device comprising a coating or printing ink containing the formulation of the present subject matter.

In an embodiment, the present subject matter is directed to a coating or printing ink comprising the formulation of the present subject matter.

In an embodiment, the present subject matter is directed to an organic electronic (OE) device prepared from the formulation of the present subject matter.

In an embodiment, the present subject matter is directed to a synthesis of monomers comprising one or more of the following steps:

reacting 4,5-difluoro-2-nitroaniline (Compound 1) with a base, for example sodium hydroxide or potassium hydroxide, in an organic solvent mixture containing solvents, for example tetrahydrofuran or 1,4-dioxane, via a ring-closure reaction, to produce 5,6-difluoro-2,1,3-benzoxadiazole 1-oxide (Compound 2);

reacting Compound 2 with a reductant, for example triethyl phosphite or triphenyl phosphite, in an organic solvent mixture containing solvents, for example tetrahydrofuran or toluene, via a reduction reaction, to obtain 5,6-difluoro-2,1,3-benzoxadiazole (Compound 3);

reacting Compound 3 with a Lewis acid, for example trimethylsilyl chloride, and a base, for example lithium diisopropylamide, in an organic solvent mixture containing solvents, for example tetrahydrofuran, via a substitution reaction, to obtain 5,6-difluoro-4,7-bis(trimethylsilyl)-2,1,3-benzoxadiazole (Compound 4); and

reacting Compound 4 with a bromination reagent, for example N-bromosuccinimide, in an organic solvent mixture containing solvents, for example sulfuric acid, via a substitution reaction, to obtain 4,7-dibromo-5,6-difluoro-2,1,3-benzoxadiazole (Compound 5).

In an embodiment, the present subject matter is directed to a monomer prepared according to the aforementioned synthesis.

In an embodiment, the present subject matter is directed to a synthesis of monomers comprising one or more of the following steps:

reacting 4-fluoro-2-nitroaniline (Compound 8) with a base, for example sodium hydroxide or potassium hydroxide, in an organic solvent mixture containing solvents, for example tetrahydrofuran or 1,4-dioxane, via a ring-closure reaction to obtain 5-fluoro-2,1,3-benzoxadiazole 1-oxide (Compound 9);

reacting Compound 9 with a reductant, for example triethyl phosphite or triphenyl phosphite, in an organic solvent mixture containing solvents, for example tetrahydrofuran or toluene, via a reduction reaction, to obtain 5-fluoro-2,1,3-benzoxadiazole (Compound 10);

reacting Compound 10 with a Lewis acid, for example trimethylsilyl chloride, and a base, for example lithium diisopropylamide, in an organic solvent mixture containing solvents, for example tetrahydrofuran, via a substitution reaction, to obtain 5-fluoro-4,7-bis(trimethylsilyl)-2,1,3-benzoxadiazole (Compound 11); and

reacting Compound 11 with a bromination reagent, for example N-bromosuccinimide, in an organic solvent mixture containing solvents, for example sulfuric acid, via a substitution reaction to obtain 4,7-dibromo-5,6-difluoro-2,1,3-benzoxadiazole (Compound 12).

In an embodiment, the present subject matter is directed to a monomer prepared according to the aforementioned synthesis.

### BRIEF DESCRIPTION OF THE DRAWINGS

FIG. 1 shows the UV-Vis spectra of a polymer in thin film according to one embodiment of the present subject matter.

FIG. 2 shows a comparison plot of Current and Potential vs. Fc/Fc⁺ of PffBX4T-2DT. The scan rate is 0.1 V s⁻¹.

FIG. 3 shows A) current-voltage and B) EQE curves of an optimized PffBX4T-2DT:PC₇₁BM solar cell.

### DETAILED DESCRIPTION

### Definitions

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include two or more heteroaryl rings fused together and monocyclic heteroaryl rings fused to one or more aromatic carbocyclic rings, non-aromatic carbocyclic rings, and/or non-aromatic cycloheteroalkyl rings. A heteroaryl group, as a whole, can have, for example, 5 to 22 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5- or 6-membered monocyclic and 5-6 bicyclic ring systems shown below:

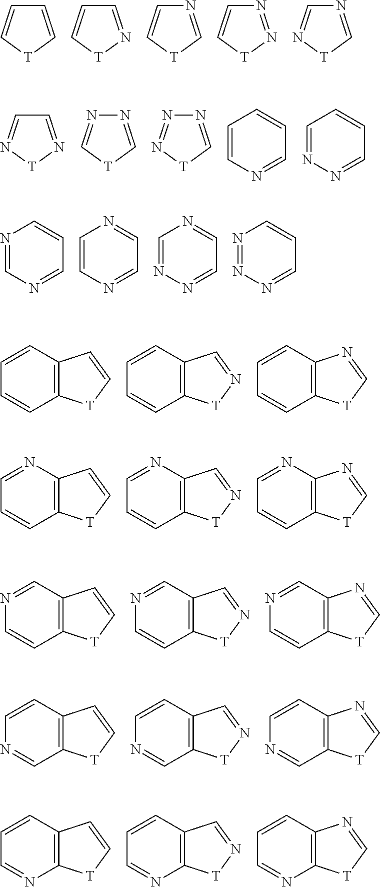

where T is O, S, NH, N-alkyl, N-aryl, N-(arylalkyl) (e.g., N-benzyl), SiH₂, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyridyl, pyrim-idyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, ben-zothienyl, quinolyl, 2-methylquinolyl, isoquinolyl, quinox-alyl, quinazolyl, benzotriazolyl, benzimidazolyl, benzothia-zolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pte-ridinyl, purinyl, oxazolopyridinyl, thiazolopyridinyl, imida-zopyridinyl, furopyridinyl, thienopyridinyl, pyridopyrimidi-nyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofu-ropyridinyl groups, and the like. In some embodiments, heteroaryl groups can be substituted as described herein.

As used herein, a “p-type semiconductor material” or a “donor” material refers to a semiconductor material, for example, an organic semiconductor material, having holes as the majority current or charge carriers. In some embodiments, when a p-type semiconductor material is deposited on a substrate, it can provide a hole mobility in excess of about 10⁻⁵ cm²/Vs. In the case of field-effect devices, a p-type semiconductor also can exhibit a current on/off ratio of greater than about 10.

As used herein, an “n-type semiconductor material” or an “acceptor” material refers to a semiconductor material, for example, an organic semiconductor material, having electrons as the majority current or charge carriers. In some embodiments, when an n-type semiconductor material is deposited on a substrate, it can provide an electron mobility in excess of about 10⁻⁵ cm²/Vs. In the case of field-effect devices, an n-type semiconductor also can exhibit a current on/off ratio of greater than about 10.

As used herein, “mobility” refers to a measure of the velocity with which charge carriers, for example, holes (or units of positive charge) in the case of a p-type semiconductor material and electrons (or units of negative charge) in the case of an n-type semiconductor material, move through the material under the influence of an electric field. This parameter, which depends on the device architecture, can be measured using a field-effect device or space-charge limited current measurements.

As used herein, a compound can be considered “ambient stable” or “stable at ambient conditions” when a transistor incorporating the compound as its semiconducting material exhibits a carrier mobility that is maintained at about its initial measurement when the compound is exposed to ambient conditions, for example, air, ambient temperature, and humidity, over a period of time. For example, a compound can be described as ambient stable if a transistor incorporating the compound shows a carrier mobility that does not vary more than 20% or more than 10% from its initial value after exposure to ambient conditions, including, air, humidity and temperature, over a 3 day, 5 day, or 10 day period.

As used herein, fill factor (FF) is the ratio (given as a percentage) of the actual maximum obtainable power, (Pm or Vmp*Jmp), to the theoretical (not actually obtainable) power, (Jsc*Voc). Accordingly, FF can be determined using the equation:

FF=(Vmp*Jmp)/(Jsc*Voc)

where Jmp and Vmp represent the current density and voltage at the maximum power point (Pm), respectively, this point being obtained by varying the resistance in the circuit until J*V is at its greatest value; and Jsc and Voc represent the short circuit current and the open circuit voltage, respectively. Fill factor is a key parameter in evaluating the performance of solar cells. Commercial solar cells typically have a fill factor of about 0.60% or greater.

As used herein, the open-circuit voltage (Voc) is the difference in the electrical potentials between the anode and the cathode of a device when there is no external load connected.

As used herein, the power conversion efficiency (PCE) of a solar cell is the percentage of power converted from absorbed light to electrical energy. The PCE of a solar cell can be calculated by dividing the maximum power point (Pm) by the input light irradiance (E, in W/m2) under standard test conditions (STC) and the surface area of the solar cell (Ac in m2). STC typically refers to a temperature of 25° C. and an irradiance of 1000 W/m2 with an air mass 1.5 (AM 1.5) spectrum.

As used herein, a component (such as a thin film layer) can be considered “photoactive” if it contains one or more compounds that can absorb photons to produce excitons for the generation of a photocurrent.

As used herein, “solution-processable” refers to compounds (e.g., polymers), materials, or compositions that can be used in various solution-phase processes including spin-coating, printing (e.g., inkjet printing, gravure printing, offset printing and the like), spray coating, electrospray coating, drop casting, dip coating, blade coating, and the like.

As used herein, a “semicrystalline polymer” refers to a polymer that has an inherent tendency to crystallize at least partially either when cooled from a melted state or deposited from solution, when subjected to kinetically favorable conditions such as slow cooling, or low solvent evaporation rate and so forth. The crystallization or lack thereof can be readily identified by using several analytical methods, for example, differential scanning calorimetry (DSC) and/or X-ray diffraction (XRD).

As used herein, “annealing” refers to a post-deposition heat treatment to the semicrystalline polymer film in ambient or under reduced/increased pressure for a time duration of more than 100 seconds, and “annealing temperature” refers to the maximum temperature that the polymer film is exposed to for at least 60 seconds during this process of annealing. Without wishing to be bound by any particular theory, it is believed that annealing can result in an increase of crystallinity in the polymer film, where possible, thereby increasing field effect mobility. The increase in crystallinity can be monitored by several methods, for example, by comparing the differential scanning calorimetry (DSC) or X-ray diffraction (XRD) measurements of the as-deposited and the annealed films.

As used herein, a “polymeric compound” (or “polymer”) refers to a molecule including a plurality of one or more repeating units connected by covalent chemical bonds. A polymeric compound can be represented by General Formula I:

*-(-(Ma)(Mb)_{y}-)_{z}* General Formula I

wherein each Ma and Mb is a repeating unit or monomer. The polymeric compound can have only one type of repeating unit as well as two or more types of different repeating units. When a polymeric compound has only one type of repeating unit, it can be referred to as a homopolymer. When a polymeric compound has two or more types of different repeating units, the term “copolymer” or “copolymeric compound” can be used instead. For example, a copolymeric compound can include repeating units where Ma and Mb represent two different repeating units. Unless specified otherwise, the assembly of the repeating units in the copolymer can be head-to-tail, head-to-head, or tail-to-tail. In addition, unless specified otherwise, the copolymer can be a random copolymer, an alternating copolymer, or a block copolymer. For example, General Formula I can be used to represent a copolymer of Ma and Mb having x mole fraction of Ma and y mole fraction of Mb in the copolymer, where the manner in which comonomers Ma and Mb is repeated can be alternating, random, regiorandom, regioregular, or in blocks, with up to z comonomers present. In addition to its composition, a polymeric compound can be further characterized by its degree of polymerization (n) and molar mass (e.g., number average molecular weight (M) and/or weight average molecular weight (Mw) depending on the measuring technique(s)).

As used herein, “halo” or “halogen” refers to fluoro, chloro, bromo, and iodo.

As used herein, “alkyl” refers to a straight-chain or branched saturated hydrocarbon group. Examples of alkyl groups include methyl (Me), ethyl (Et), propyl (e.g., n-propyl and z′-propyl), butyl (e.g., n-butyl, z′-butyl, sec-butyl, tert-butyl), pentyl groups (e.g., n-pentyl, z′-pentyl, -pentyl), hexyl groups, and the like. In various embodiments, an alkyl group can have 1 to 40 carbon atoms (i.e., C1-40 alkyl group), for example, 1-30 carbon atoms (i.e., C1-30 alkyl group). In some embodiments, an alkyl group can have 1 to 6 carbon atoms, and can be referred to as a “lower alkyl group.” Examples of lower alkyl groups include methyl, ethyl, propyl (e.g., n-propyl and z′-propyl), and butyl groups (e.g., n-butyl, z′-butyl, sec-butyl, ten-butyl). In some embodiments, alkyl groups can be substituted as described herein. An alkyl group is generally not substituted with another alkyl group, an alkenyl group, or an alkynyl group.

As used herein, “alkenyl” refers to a straight-chain or branched alkyl group having one or more carbon-carbon double bonds. Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In various embodiments, an alkenyl group can have 2 to 40 carbon atoms (i.e., C2-40 alkenyl group), for example, 2 to 20 carbon atoms (i.e., C2-20 alkenyl group). In some embodiments, alkenyl groups can be substituted as described herein. An alkenyl group is generally not substituted with another alkenyl group, an alkyl group, or an alkynyl group.

As used herein, a “fused ring” or a “fused ring moiety” refers to a polycyclic ring system having at least two rings where at least one of the rings is aromatic and such aromatic ring (carbocyclic or heterocyclic) has a bond in common with at least one other ring that can be aromatic or non-aromatic, and carbocyclic or heterocyclic. These polycyclic ring systems can be highly p-conjugated and optionally substituted as described herein.

As used herein, “heteroatom” refers to an atom of any element other than carbon or hydrogen and includes, for example, nitrogen, oxygen, silicon, sulfur, phosphorus, and selenium.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have 6 to 24 carbon atoms in its ring system (e.g., C6-24 aryl group), which can include multiple fused rings. In some embodiments, a polycyclic aryl group can have 8 to 24 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In some embodiments, aryl groups can be substituted as described herein. In some embodiments, an aryl group can have one or more halogen substituents, and can be referred to as a “haloaryl” group. Perhaloaryl groups, i.e., aryl groups where all of the hydrogen atoms are replaced with halogen atoms (e.g., —C6F5), are included within the definition of “haloaryl.” In certain embodiments, an aryl group is substituted with another aryl group and can be referred to as a biaryl group. Each of the aryl groups in the biaryl group can be substituted as disclosed herein.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, can have, for example, 5 to 24 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine Noxide thiophene S-oxide, thiophene S,S-dioxide). Examples of heteroaryl groups include, for example, the 5- or 6-membered monocyclic and 5-6 bicyclic ring systems shown below: where T is O, S, NH, N-alkyl, N-aryl, N-(arylalkyl) (e.g., N-benzyl), SiH2, SiH(alkyl), Si(alkyl)2, SiH(arylalkyl), Si(arylalkyl)2, or Si(alkyl)(arylalkyl). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In some embodiments, heteroaryl groups can be substituted as described herein.

### Abbreviations

BHJ bulk-heterojunction
BT benzothiadiazole
BX benzoxadiazole
DSC differential scanning calorimetry
EQE external quantum efficiency
FF fill factor
ffBT difluorobenzothiadiazole
IC integrated circuit
ITO indium tin oxide
O-SC organic solar cell
OE organic electronic
OFET organic field-effect transistor
O-IC organic integrated circuit
OLED organic light emitting diode
OLET organic light emitting transistor
OPV organic photovoltaic
OSC organic semiconductor
PC₇₁BM phenyl-C₇₁-butyric-acid-methyl-ester
PCE power conversion efficiency
RFID radio frequency identification
STC standard test conditions
TFT thin film transistor
THF tetrahydrofuran
UV ultraviolet
XRD x-ray diffraction

### Polymer

In an embodiment, the present subject matter is directed to a polymer comprising one or more repeating units of formula I:

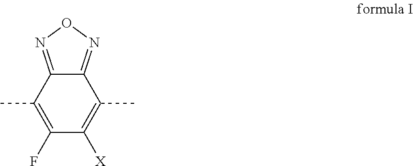

wherein X is H or F.

In an embodiment, the units of formula I are selected from formulae II and III:

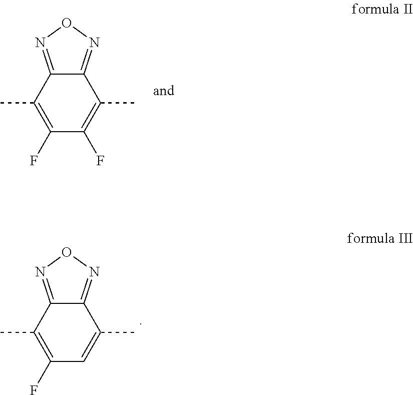

In an embodiment, the polymer of the present subject matter is characterized in that it comprises one or more repeating units of formula IV:

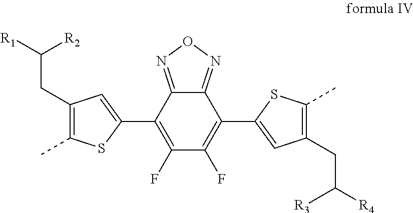

wherein R₁, R₂, R₃ and R₄, at each occurrence, independently can be a C₁₋₄₀ alkyl group.

In an embodiment, the polymer of the present subject matter is characterized in that it comprises one or more repeating units of formula V:

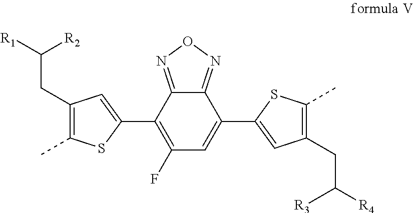

wherein R₁, R₂, R₃ and R₄, at each occurrence, independently can be a C₁₋₄₀ alkyl group.

In an embodiment, the polymer of the present subject matter is characterized in that it comprises one or more repeating units of formula VI:

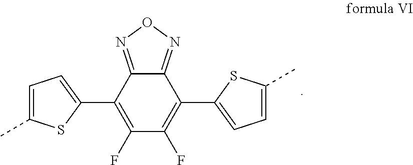

In an embodiment, the polymer of the present subject matter is characterized in that it comprises one or more repeating units of formula VII:

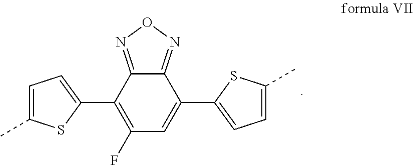

In an embodiment, the polymer of the present subject matter is characterized in that it comprises one or more repeating units of formula X:

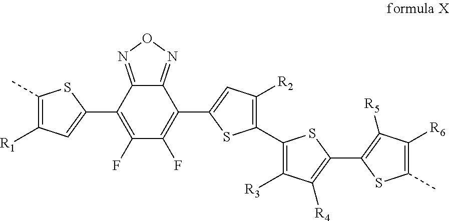

wherein R₁, R₂, R₃, R₄, R₅ and R₆, at each occurrence, independently can be a C₁₋₄₀ alkyl group.

In an embodiment, the polymer of the present subject matter is characterized in that it comprises one or more repeating units of formula XI:

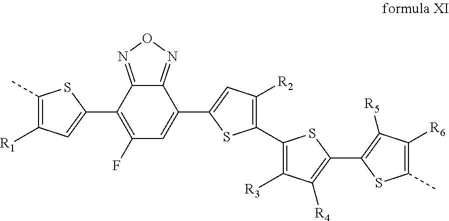

wherein R₁, R₂, R₃, R₄, R₅ and R₆, at each occurrence, independently can be a C₁₋₄₀ alkyl group.

In an embodiment, the polymer of the present subject matter is characterized in that it comprises one or more repeating units of formula XII:

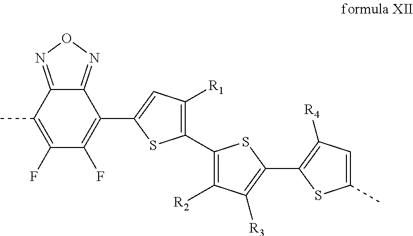

wherein R₁, R₂, R₃ and R₄, at each occurrence, independently can be a C₁₋₄₀ alkyl group.

In an embodiment, the polymer of the present subject matter is characterized in that it comprises one or more repeating units of formula XIII:

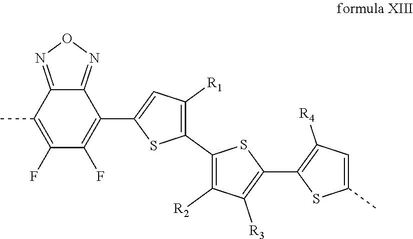

wherein R₁, R₂, R₃ and R₄, at each occurrence, independently can be a C₁₋₄₀ alkyl group.

In an embodiment, the polymer of the present subject matter is characterized in that it comprises one or more repeating units selected from:

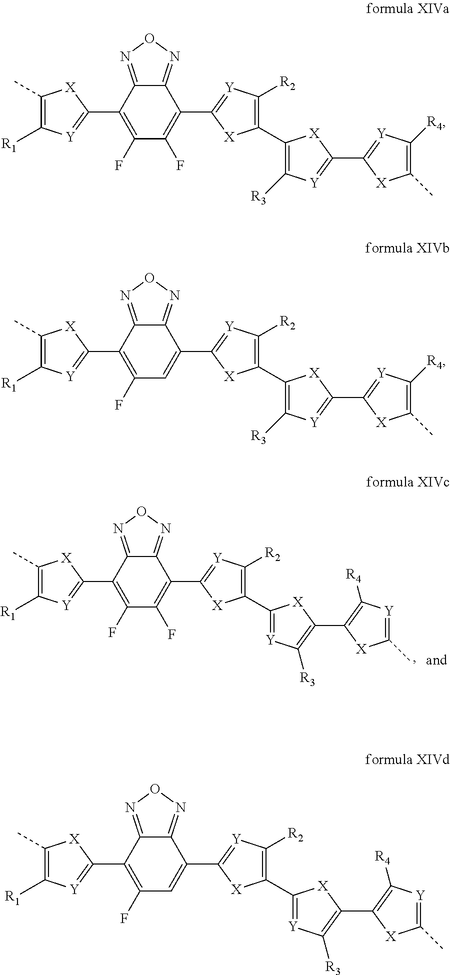

wherein
R₁, R₂, R₃ and R₄, at each occurrence, independently can be a C1-40 alkyl group; each X is independently selected from the group consisting of O, S, Se and Te; and each Y is independently selected from the group consisting of N, C—H, and C—R5,
   wherein R5 is selected from the group consisting of C1-40 straight-chain and branched alkyl groups.

In an embodiment, the polymer of the present subject matter is characterized in that it comprises one or more repeating units selected from:

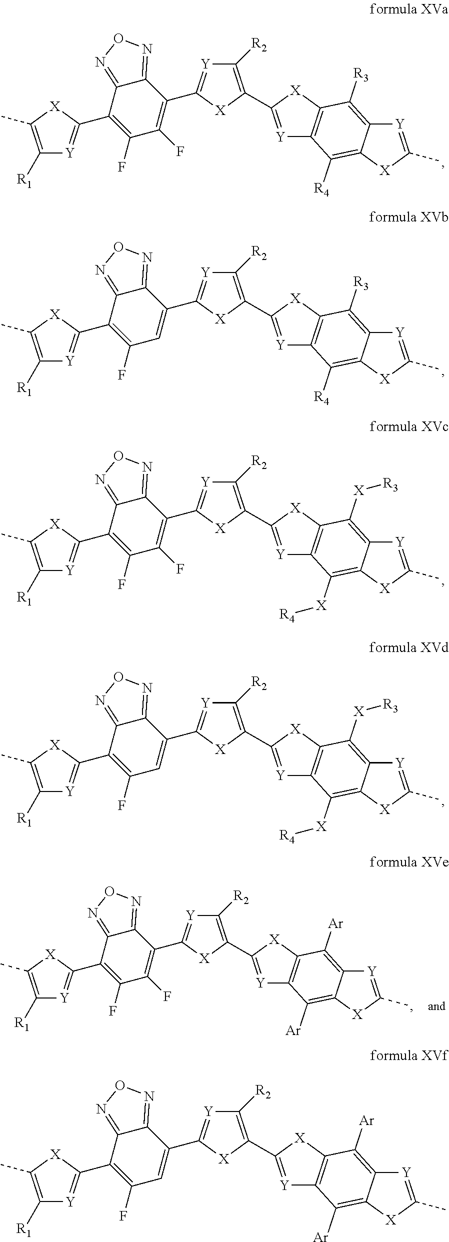

wherein

R₁, R₂, R₃ and R₄, at each occurrence, independently can be a C1-40 alkyl group;

each X is independently selected from the group consisting of O, S, Se and Te;

each Y is independently selected from the group consisting of N, C—H, and C—R5,

wherein R5 is selected from the group consisting of C1-40 straight-chain and branched alkyl groups; and

each Ar is independently selected from the group consisting of unsubstituted or substituted monocyclic, bicyclic, and polycyclic arylene, and monocyclic, bicyclic, and polycyclic heteroarylene, wherein each Ar may contain one to five of said arylene or heteroarylene each of which may be fused or linked.

In an embodiment, the polymer of the present subject matter is characterized in that it comprises one or more repeating units selected from:

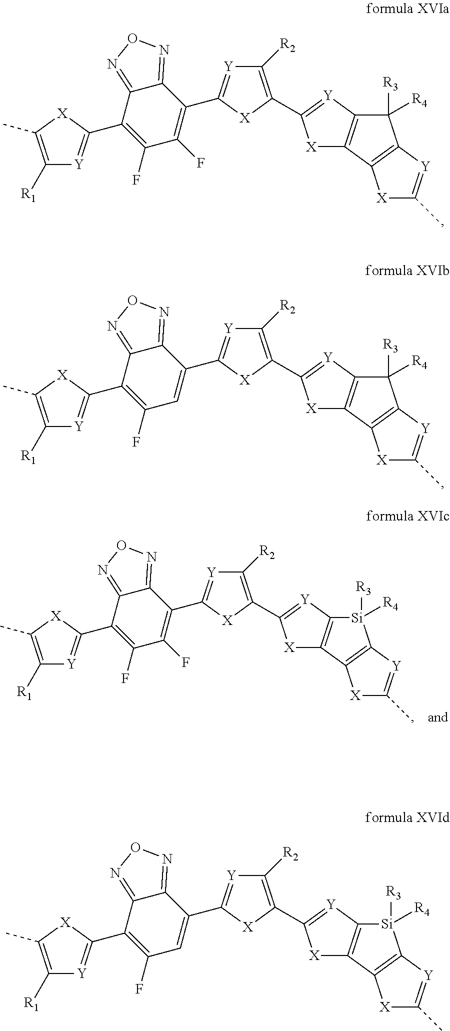

wherein

R₁, R₂, R₃ and R₄, at each occurrence, independently can be a C1-40 alkyl group;

each X is independently selected from the group consisting of O, S, Se and Te; and

each Y is independently selected from the group consisting of N, C—H, and C—R5,

wherein R5 is selected from the group consisting of C1-40 straight-chain and branched alkyl groups.

In an embodiment, the polymer of the present subject matter is characterized in that it comprises one or more repeating units selected from:

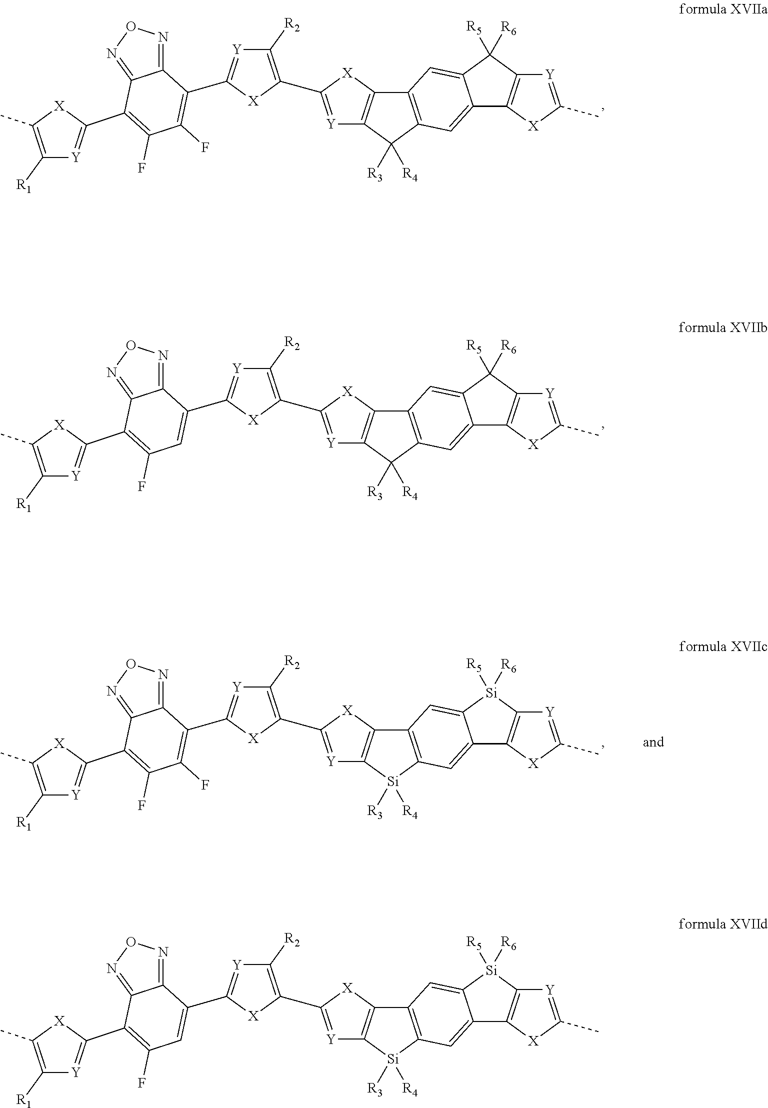

wherein

R₁, R₂, R₃, R₄, R₅ and R₆, at each occurrence, independently can be a C1-40 alkyl group;

each X is independently selected from the group consisting of O, S, Se and Te; and

each Y is independently selected from the group consisting of N, C—H, and C—R5,

wherein R5 is selected from the group consisting of C1-40 straight-chain and branched alkyl groups.

In an embodiment, the one or more repeating units of formula I has formula II:

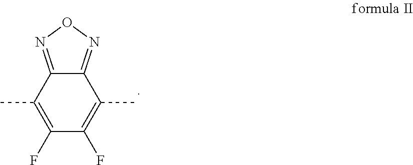

In an embodiment, the one or more repeating units of formula I has formula III:

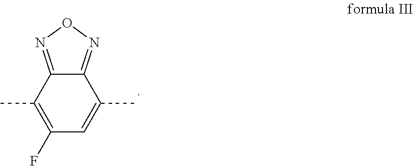

In an embodiment, the one or more repeating units of formula I comprises one or more repeating units of formula IV:

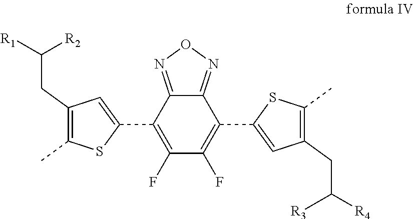

wherein R₁, R₂, R₃ and R₄, at each occurrence, independently can be a C₁₋₄₀ alkyl group.

In an embodiment, the one or more repeating units of formula I comprises one or more repeating units of formula V:

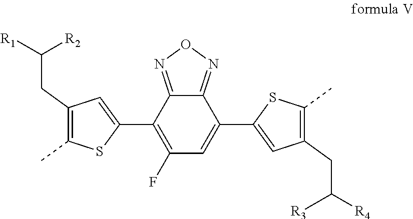

wherein R₁, R₂, R₃ and R₄, at each occurrence, independently can be a C₁₋₄₀ alkyl group.

In an embodiment, the one or more repeating units of formula I comprises one or more repeating units of formula VI:

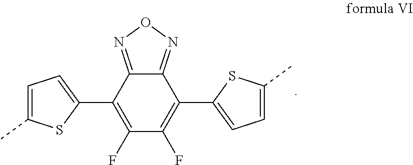

In an embodiment, the one or more repeating units of formula I comprises one or more repeating units of formula VII:

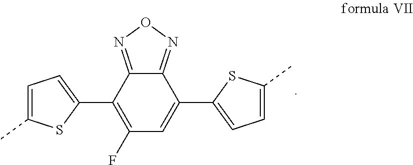

In an embodiment, the one or more repeating units of formula I comprises one or more repeating units of formula X:

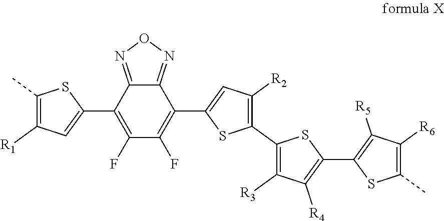

wherein R₁, R₂, R₃, R₄, R₅ and R₆, at each occurrence, independently can be a C₁₋₄₀ alkyl group.

In an embodiment, the one or more repeating units of formula I comprises one or more repeating units of formula XI:

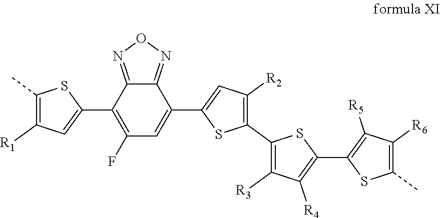

wherein R₁, R₂, R₃, R₄, R₅ and R₆, at each occurrence, independently can be a C₁₋₄₀ alkyl group.

In an embodiment, the one or more repeating units of formula I comprises one or more repeating units of formula XII:

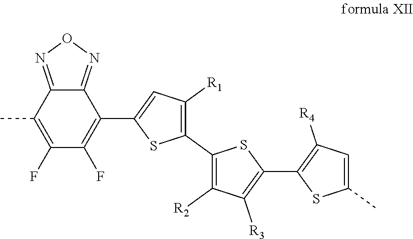

wherein R₁, R₂, R₃ and R₄, at each occurrence, independently can be a C₁₋₄₀ alkyl group.

In an embodiment, the one or more repeating units of formula I comprises one or more repeating units of formula XIII:

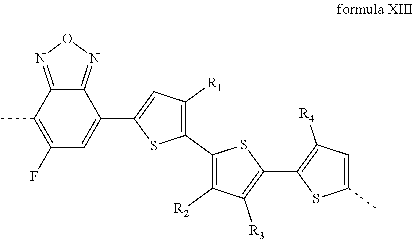

wherein R₁, R₂, R₃ and R₄, at each occurrence, independently can be a C₁₋₄₀ alkyl group.

In an embodiment, the one or more repeating units of formula I comprises one or more repeating units selected from the group consisting of

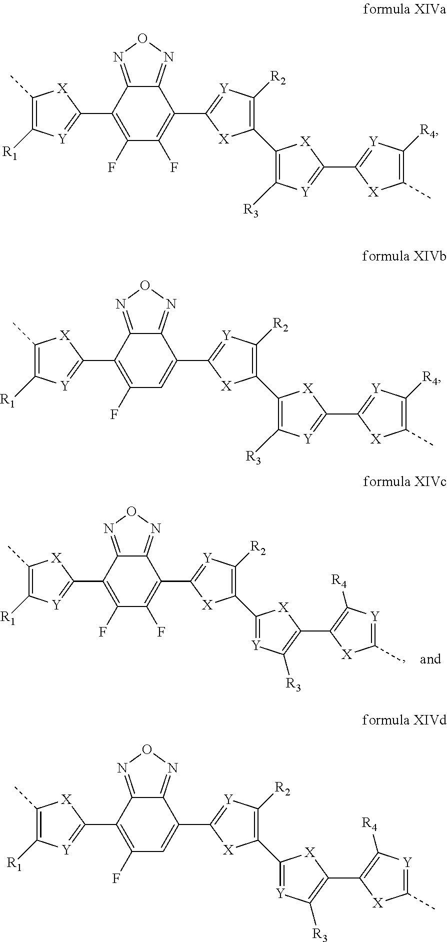

wherein

R₁, R₂, R₃ and R₄, at each occurrence, independently can be a C1-40 alkyl group;

each X is independently selected from the group consisting of O, S, Se and Te; and

each Y is independently selected from the group consisting of N, C—H, and C—R5,

wherein R5 is selected from the group consisting of C1-40 straight-chain and branched alkyl groups.

In an embodiment, the one or more repeating units of formula I comprises one or more repeating units selected from the group consisting of

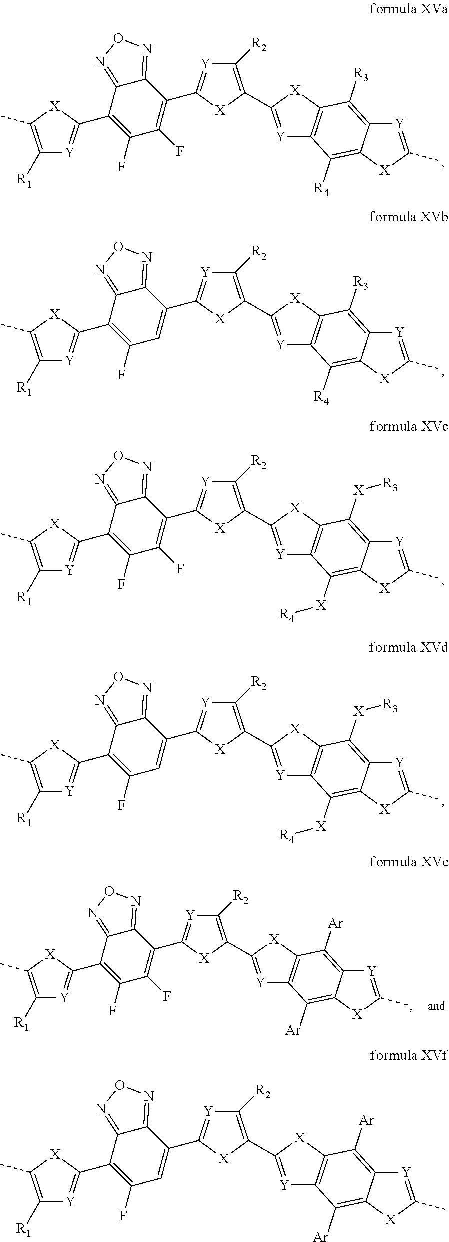

wherein

R₁, R₂, R₃ and R₄, at each occurrence, independently can be a C1-40 alkyl group;

each X is independently selected from the group consisting of O, S, Se and Te;

each Y is independently selected from the group consisting of N, C—H, and C—R5, wherein R5 is selected from the group consisting of C1-40 straight-chain and branched alkyl groups; and

each Ar is independently selected from the group consisting of unsubstituted or substituted monocyclic, bicyclic, and polycyclic arylene, and monocyclic, bicyclic, and polycyclic heteroarylene, wherein each Ar may contain one to five of said arylene or heteroarylene each of which may be fused or linked.

In an embodiment, the one or more repeating units of formula I comprises one or more repeating units selected from the group consisting of

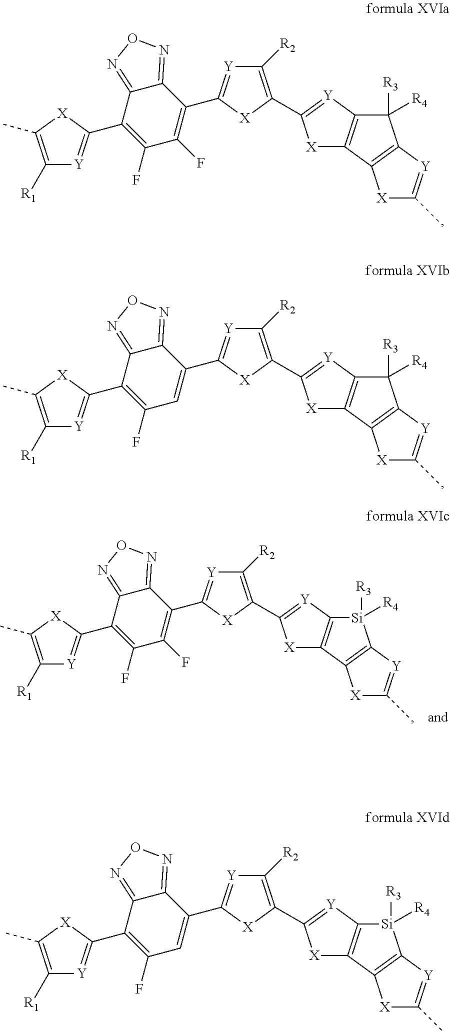

wherein

R₁, R₂, R₃ and R₄, at each occurrence, independently can be a C1-40 alkyl group;

each X is independently selected from the group consisting of O, S, Se and Te; and

each Y is independently selected from the group consisting of N, C—H, and C—R5,

wherein R5 is selected from the group consisting of C1-40 straight-chain and branched alkyl groups.

In an embodiment, the one or more repeating units of formula I comprises one or more repeating units selected from the group consisting of

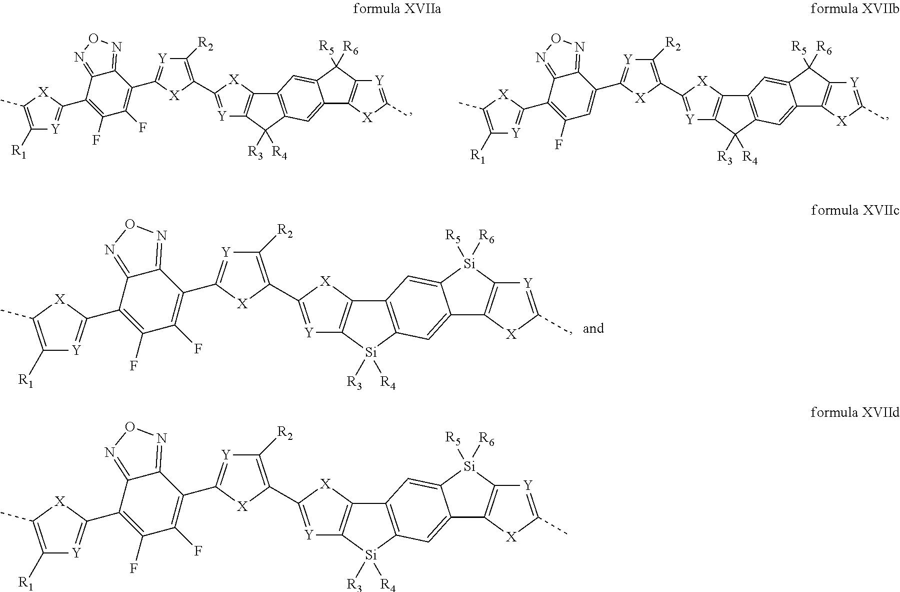

wherein

R₁, R₂, R₃, R₄, R₅ and R₆, at each occurrence, independently can be a C1-40 alkyl group;

each X is independently selected from the group consisting of O, S, Se and Te; and

each Y is independently selected from the group consisting of N, C—H, and C—R5,

wherein R5 is selected from the group consisting of C1-40 straight-chain and branched alkyl groups.

In an embodiment, the present subject matter is directed to a process of preparing a polymer or organic compound comprising polymerizing an intermediate with formula VIII:

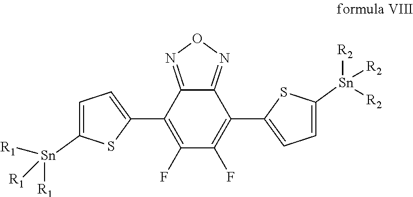

wherein R₁ and R₂, at each occurrence, independently can be a C₁₋₁₀ alkyl group.

In an embodiment, the present subject matter is directed to a process of preparing a polymer or organic compound comprising polymerizing an intermediate with formula IX:

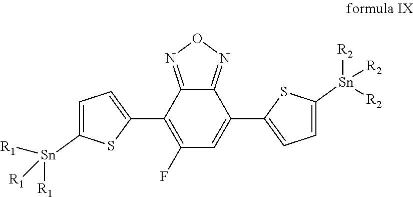

wherein R₁ and R₂, at each occurrence, independently can be a C₁₋₁₀ alkyl group.

In an embodiment, the present subject matter is directed to a conjugated polymer comprising a repeating unit (Ml), wherein Ml has a formula of:

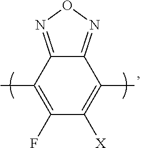

wherein X is H or F.

In an embodiment, the conjugated polymer of the present subject matter further comprises one or more repeating units other than M1. For example, the one or more repeating units (M2) may be selected from:

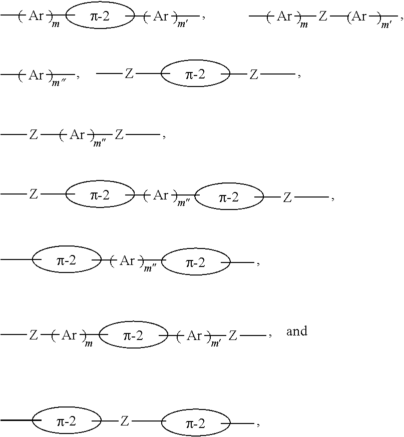

wherein

each π-2 is independently an optionally substituted fused ring moiety;

each Ar is independently an optionally substituted 5- or 6-membered aryl or heteroaryl group;

each Z is independently a conjugated linear linker; and

each m, m′, and m″, independently=0, 1, 2, 3, 4, 5, or 6.

In certain embodiments, π-2 can have a reduction potential greater than or equal to about −2.2 V. In particular embodiments, π-2 can have a reduction potential greater than or equal to about −1.2 V. Examples of suitable cyclic cores include naphthalene, anthracene, tetracene, pentacene, perylene, pyrene, coronene, fluorene, indacene, inde-nofluorene, and tetraphenylene, as well as their analogs in which one or more carbon atoms can be replaced with a heteroatom such as O, S, Si, Se, N, or P. In certain embodiments, π-2 can include at least one electron-withdrawing group.

In certain embodiments, π-2 can include two or more (e.g., 2-4) fused rings where each ring can be an optionally substituted five-, six-, or seven-membered ring. In some embodiments, π-2 can include a monocyclic ring (e.g., a 1,3-dioxolane group or a derivative thereof including optional substituents and/or ring heteroatoms) covalently bonded to a second monocyclic ring or a polycyclic system via a spiro atom (e.g., a spiro carbon atom).

In certain embodiments, π-2 can include two or more (e.g., 2-4) fused rings where each ring can be an optionally substituted five-, six-, or seven-membered ring. In some embodiments, π-2 can include a monocyclic ring (e.g., a 1,3-dioxolane group or a derivative thereof including optional substituents and/or ring heteroatoms) covalently bonded to a second monocyclic ring or a polycyclic system via a spiro atom (e.g., a spiro carbon atom).

In some embodiments, π-2 is selected from the group consisting of:

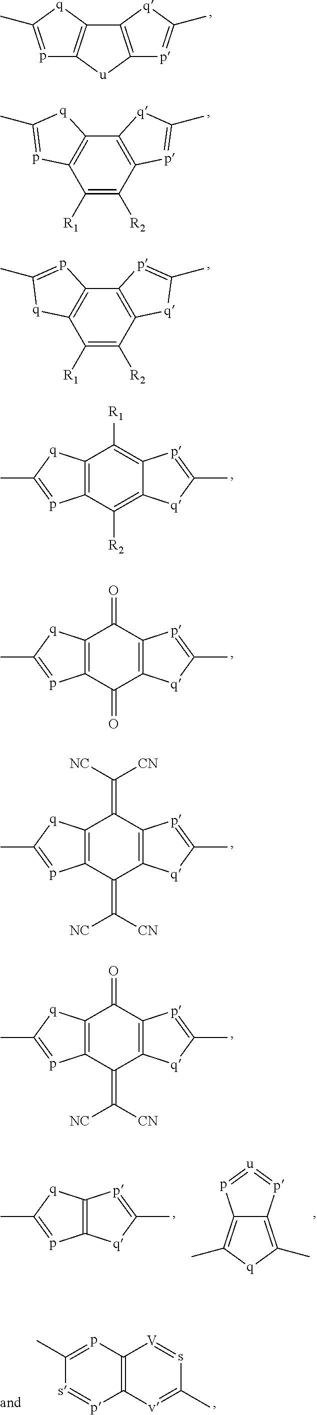

wherein

p, p′, s, s′, v, and v′ independently can be selected from ═CR₁—, ═N—, and ═SiR₁—;

q, q′, and u independently can be selected from —C(O)—, —C(C(CN)₂)—, —S—, S(O)—, —S(O)₂, —O—, —SiR₁R₂—, —CR₁R₂—, —CR₁R₂—CR₁R₂—, and —CR₁═CR₂—; and

R₁ and R₂, at each occurrence, independently can be H, halogen, CN, a C₁₋₄₀ alkyl group, a C₁₋₄₀ alkoxy group, a C₁₋₄₀ alkylthio group, a C₁₋₄₀ haloalkyl group, a C₆₋₁₄ aryl group, a 5-14 membered heteroaryl group, —(OCH₂CH₂)ₜOR^{e}, —(OCF₂CF₂)ₜOR^{e}, —(OCH₂CF₂)ₜOR^{e}, —(OCF₂CH₂)ᵣOR^{e}, —(CH₂CH₂O)ᵣR^{e}, —(CF₂CF₂O)ᵣR^{e}, —(CH₂CF₂O)ᵣR^{e}, or —(CF₂CH₂O)ᵣR^{e}; wherein the C₆₋₁₄ aryl group and the 5-14 membered heteroaryl group optionally can be substituted with 1-4 groups independently selected from halogen, CN, a C₁₋₄₀ alkyl groups, a C₁₋₄₀ alkoxy group, a C₁₋₄₀ alkylthio group, and a C₁₋₄₀ haloalkyl group; t is 1, 2, 3, 4, 5, 6, 7, 8, 9 or 10; and R^{e} is a C₁₋₂₀ alkyl group or a C₁₋₂₀ haloalkyl group; and b is 1, 2, 3 or 4.

In various embodiments, the linker Z can be a conjugated system by itself (e.g., including two or more double or triple bonds) or can form a conjugated system with its neighboring components. For example, in embodiments where Z is a linear linker, Z can be a divalent ethenyl group (i.e., having one double bond), a divalent ethynyl group (i.e., having one tripe bond), a C₄₋₄₀ alkenyl or alkynyl group that includes two or more conjugated double or triple bonds, or some other non-cyclic conjugated systems that can include heteroatoms such as Si, N, P, and the like. For example, in some embodiments, Z is selected from the group consisting of:

wherein R₄ can be independently selected from H, a halogen, —CN, a C₁₋₂₀ alkyl group, a C₁₋₂₀ alkoxy group, and a C₁₋₂₀ haloalkyl group.

In an embodiment, the conjugated polymer of the present subject matter has an average molecular weight in a range from 10,000 to 1,000,000 gram/mole. In an embodiment, the conjugated polymer has an optical bandgap of 1.65 eV or lower.

In an embodiment, the conjugated polymer of the present subject matter is selected from the group consisting of:

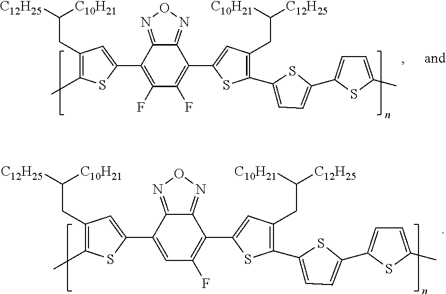

In an embodiment, a power conversion efficiency of the conjugated polymer of the present subject matter with phenyl-C₇₁-butyric-acid-methyl-ester (PC₇₁BM) is in a range between 5.0 and 15.0%. In an embodiment, a fill factor of the conjugated polymer of the present subject matter with phenyl-C₇₁-butyric-acid-methyl-ester (PC₇₁BM) is in a range between 0.60 and 0.80.

### Formulation

In an embodiment, the present subject matter is directed to a formulation comprising the polymer of the present subject matter, and a fullerene, a second polymer, or a small molecule.

In an embodiment, the present subject matter is directed to an organic electronic (OE) device comprising a coating or printing ink containing the formulation of the present subject matter. In an embodiment, the OE device is an organic field effect transistor (OFET) device. In an embodiment, the OE device is an organic photovoltaic (OPV) device.

In an embodiment, the present subject matter is directed to a coating or printing ink comprising the formulation of the present subject matter. In an embodiment, the coating or printing ink is for preparing OE devices and rigid or flexible OPV cells and devices.

In an embodiment, the present subject matter is directed to an organic electronic (OE) device prepared from the formulation of the present subject matter.

In an embodiment, the present subject matter relates to a formulation comprising the conjugated polymer discussed above, and a fullerene.

In an embodiment, the fullerene is substituted by one or more functional groups selected from the group consisting of:

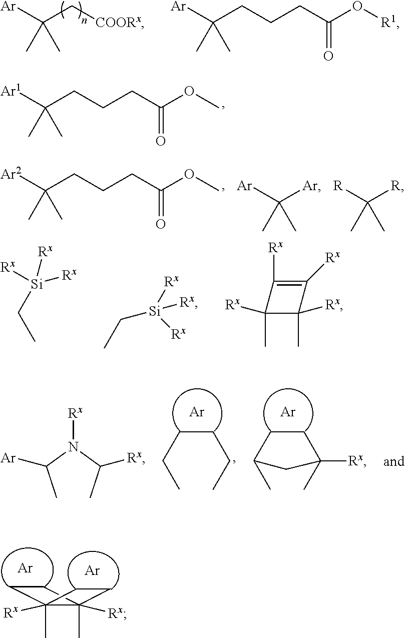

wherein

each n, independently=1-6;

each Ar is independently selected from the group consisting of monocyclic, bicyclic, and polycyclic arylene, and monocyclic, bicyclic, and polycyclic heteroarylene, wherein each Ar may contain one to five such groups, each of which may be fused or linked;

each R^{x} is independently selected from the group consisting of Ar, straight-chain, branched, and cyclic alkyl with 2-40 C atoms, wherein one or more non-adjacent C atoms are optionally replaced by —O—, —S—, —C(O)—, —C(O—)—O—, —O—C(O)—, —O—C(O)—O—, —CR⁰=CR⁰⁰—, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups, wherein R⁰ and R⁰⁰ are independently a straight-chain, branched, or cyclic alkyl group;

each R¹ is independently selected from the group consisting of straight-chain, branched, and cyclic alkyl with 2-40 C atoms, wherein one or more non-adjacent C atoms are optionally replaced by —O—, —S—, —C(O)—, —C(O)—O—, —O—C(O)—, —O—C(O)—O—, —CR⁰═CR⁰⁰—, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups, wherein the number of carbon that R¹ contains is larger than 1, wherein R⁰ and R⁰⁰ are independently a straight-chain, branched, or cyclic alkyl group;

each R is independently selected from the group consisting of straight-chain, branched, and cyclic alkyl with 2-40 C atoms, wherein one or more non-adjacent C atoms are optionally replaced by —O—, —S—, —C(O)—, —C(O—)—O—, —O—C(O)—, —O—C(O)—O—, —CR⁰=CR⁰⁰—, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups, wherein R⁰ and R⁰⁰ are independently a straight-chain, branched, or cyclic alkyl group;

each Ar¹ is independently selected from the group consisting of monocyclic, bicyclic and polycyclic heteroaryl groups, wherein each Ar¹ may contain one to five of said heteroaryl groups each of which may be fused or linked;

each Ar² is independently selected from aryl groups containing more than 6 atoms excluding H; and

wherein a fullerene ball represents a fullerene selected from the group consisting of C60, C70, C84, and other fullerenes.

In an embodiment, the fullerene is selected from the group consisting of:

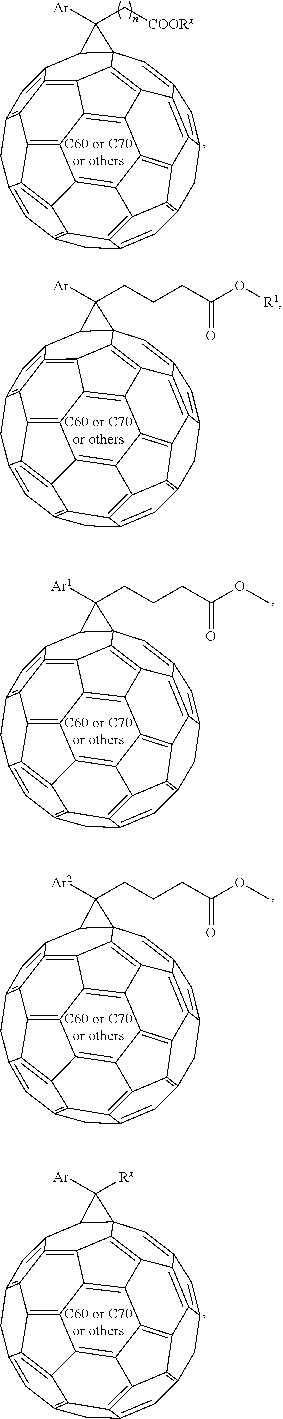

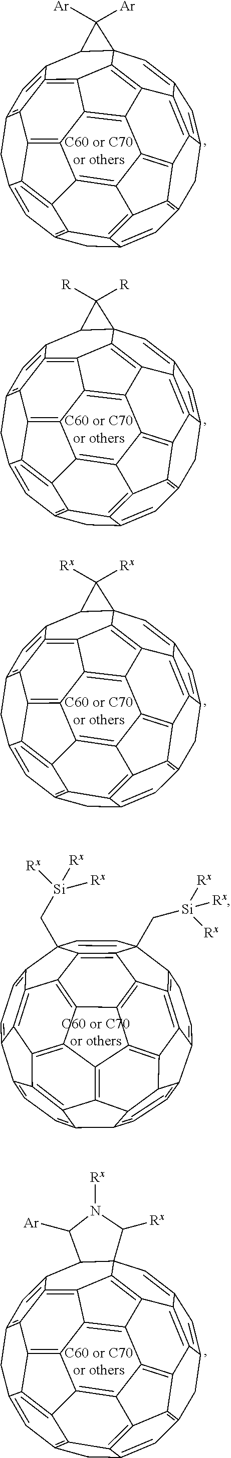

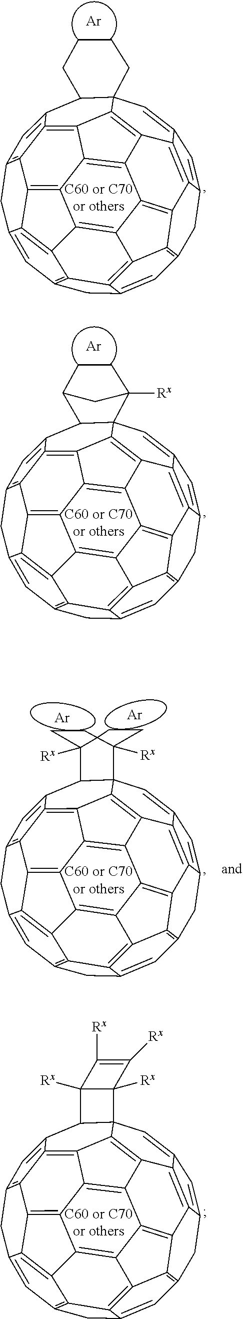

wherein each n, independently=1, 2, 4, 5, or 6.

In an embodiment, the fullerene is selected from the group consisting of:

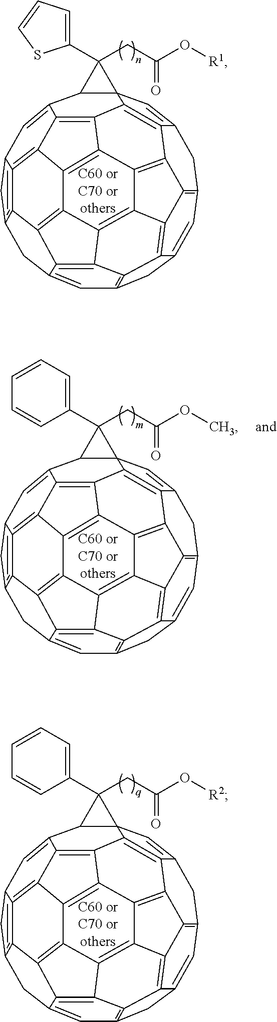

wherein

each n, independently=1-6;

each m, independently=1, 2, 4, 5, or 6;

each q, independently=1-6; and

each R¹ and R² is independently selected from the group consisting of C1-4 straight and branched chain alkyl groups; and

wherein a fullerene ball represents a fullerene from the group consisting of C60, C70, C84, and other fullerenes.

In an embodiment, the fullerene is selected from the group consisting of:

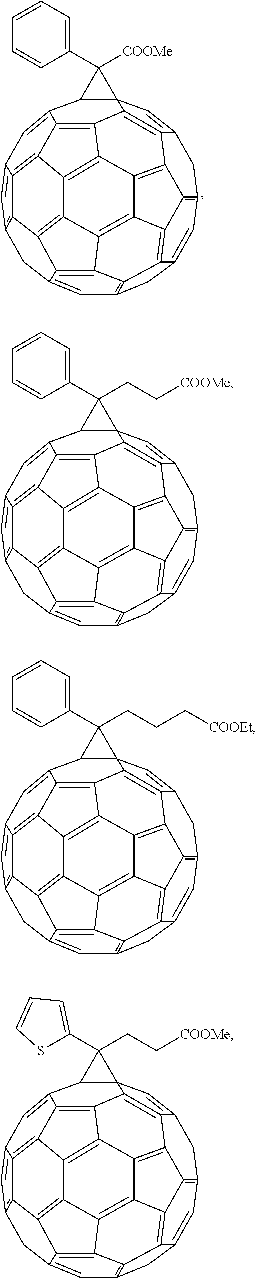

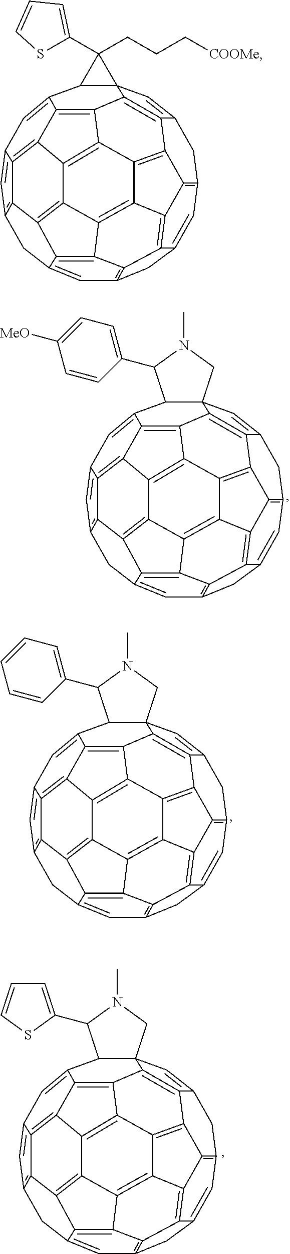

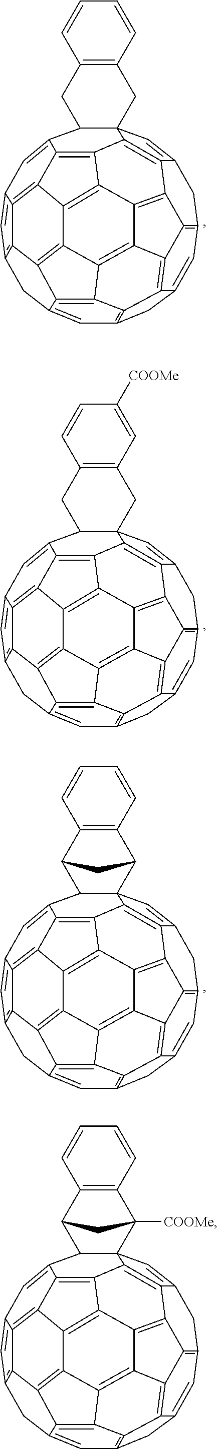

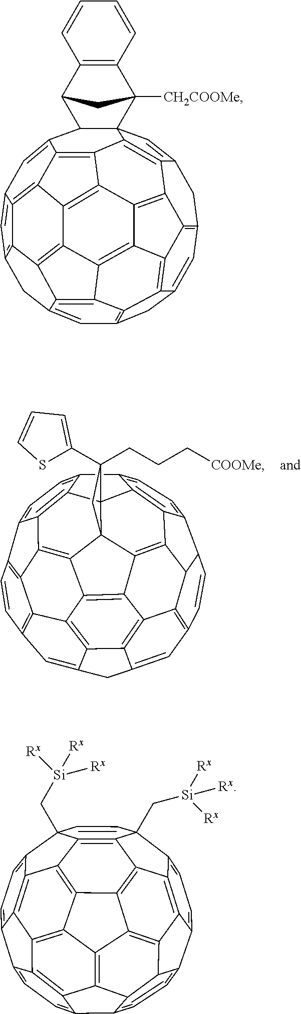

In an embodiment, the formulation of the present subject matter is a thin film.

In an embodiment, the present subject matter is directed to the formulation of the present subject matter, which further comprises an organic solvent.

In an embodiment, the present subject matter further relates to the use of the formulation as a coating or printing ink, especially for the preparation of OE devices and rigid or flexible OPV cells and devices. In an embodiment, the present subject matter further relates to an OE device prepared from the formulation. The OE devices contemplated in this regard include, without limitation, organic field effect transistors (OFET), integrated circuits (IC), thin film transistors (TFT), Radio Frequency Identification (RFID) tags, organic light emitting diodes (OLED), organic light emitting transistors (OLET), electroluminescent displays, organic photovoltaic (OPV) cells, organic solar cells (O-SC), flexible OPVs and O-SCs, organic laser diodes (O-laser), organic integrated circuits (O-IC), lighting devices, sensor devices, electrode materials, photoconductors, photodetectors, electrophotographic recording devices, capacitors, charge injection layers, Schottky diodes, planarising layers, antistatic films, conducting substrates, conducting patterns, photoconductors, electrophotographic devices, organic memory devices, biosensors and biochips.

Polymers with such structures were found to show good processability and high solubility in organic solvents, and are thus especially suitable for large scale production using solution processing methods. At the same time, the polymers show a low bandgap, high charge carrier mobility, and high external quantum efficiency in BHJ solar cells.

The formulations, methods and devices of the present subject matter provide surprising improvements in the efficiency of the OE devices and the production thereof. Unexpectedly, the performance, the lifetime and the efficiency of the OE devices can be improved, if these devices are achieved by using a formulation of the present subject matter. Furthermore, the formulation of the present subject matter provides an astonishingly high level of film forming. The homogeneity and the quality of the films can especially be improved. In addition thereto, the present subject matter enables better solution printing of OE devices, especially OPV devices.

Formulations of the present teachings can exhibit semiconductor behavior such as optimized light absorption/charge separation in a photovoltaic device; charge transport/recombination/light emission in a light-emitting device; and/or high carrier mobility and/or good current modulation characteristics in a field-effect device. In addition, the present formulations can possess certain processing advantages such as solution-processability and/or good stability (e.g., air stability) in ambient conditions. The formulations of the present teachings can be used to prepare either p-type (donor or hole-transporting), n-type (acceptor or electron-transporting), or ambipolar semiconductor materials, which in turn can be used to fabricate various organic or hybrid optoelectronic articles, structures and devices, including organic photovoltaic devices and organic light-emitting transistors.

### Synthesis

In an embodiment, the present subject matter is directed to a synthesis of monomers comprising one or more of the following steps:

reacting 4,5-difluoro-2-nitroaniline (Compound 1) with a base, for example sodium hydroxide or potassium hydroxide, in an organic solvent mixture containing solvents, for example tetrahydrofuran or 1,4-dioxane, via a ring-closure reaction, to produce 5,6-difluoro-2,1,3-benzoxadiazole 1-oxide (Compound 2);
reacting Compound 2 with a reductant, for example triethyl phosphite or triphenyl phosphite, in an organic solvent mixture containing solvents, for example tetrahydrofuran or toluene, via a reduction reaction, to obtain 5,6-difluoro-2,1,3-benzoxadiazole (Compound 3);

reacting Compound 3 with a Lewis acid, for example trimethylsilyl chloride, and a base, for example lithium diisopropylamide, in an organic solvent mixture containing solvents, for example tetrahydrofuran, via a substitution reaction, to obtain 5,6-difluoro-4,7-bis(trimethylsilyl)-2,1,3-benzoxadiazole (Compound 4); and

reacting Compound 4 with a bromination reagent, for example N-bromosuccinimide, in an organic solvent mixture containing solvents, for example sulfuric acid, via a substitution reaction, to obtain 4,7-dibromo-5,6-difluoro-2,1,3-benzoxadiazole (Compound 5).

In an embodiment, the present subject matter is directed to a monomer prepared according to the aforementioned synthesis.

In an embodiment, the present subject matter is directed to a synthesis of monomers comprising one or more of the following steps:

reacting 4-fluoro-2-nitroaniline (Compound 8) with a base, for example sodium hydroxide or potassium hydroxide, in an organic solvent mixture containing solvents, for example tetrahydrofuran or 1,4-dioxane, via a ring-closure reaction to obtain 5-fluoro-2,1,3-benzoxadiazole 1-oxide (Compound 9);

reacting Compound 9 with a reductant, for example triethyl phosphite or triphenyl phosphite, in an organic solvent mixture containing solvents, for example tetrahydrofuran or toluene, via a reduction reaction, to obtain 5-fluoro-2,1,3-benzoxadiazole (Compound 10);

reacting Compound 10 with a Lewis acid, for example trimethylsilyl chloride, and a base, for example lithium diisopropylamide, in an organic solvent mixture containing solvents, for example tetrahydrofuran, via a substitution reaction, to obtain 5-fluoro-4,7-bis(trimethylsilyl)-2,1,3-benzoxadiazole (Compound 11); and reacting Compound 11 with a bromination reagent, for example N-bromosuccinimide, in an organic solvent mixture containing solvents, for example sulfuric acid, via a substitution reaction to obtain 4,7-dibromo-5,6-difluoro-2,1,3-benzoxadiazole (Compound 12).

In an embodiment, the present subject matter is directed to a monomer prepared according to the aforementioned synthesis.

### EXAMPLES

### Example 1—Synthesis of Monomers

In an embodiment, the present subject matter is directed to synthesis of monomers according to the following synthetic route:

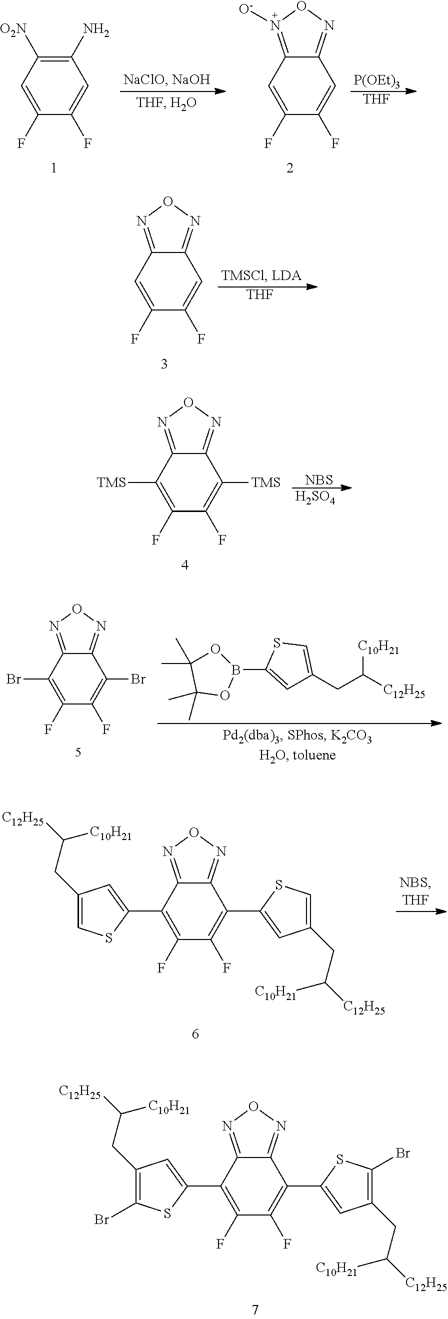

### Step 1: Preparation of 5,6-difluoro-2,1,3-benzoxadiazole 1-oxide (2)

A mixture of compound 1 (17.4 g, 100 mmol), sodium hydroxide (1.20 g, 30.0 mmol) and tetrahydrofuran (200 mL) was cooled to 0° C. Sodium hypochlorite solution (13% available chlorine, 90 mL) was added dropwise. Upon addition, the mixture was stirred at 0° C. for another 2 h. The mixture was diluted with water and extracted with dichloromethane for three times. The organic extract was combined and washed successively with water and a saturated solution of ammonium chloride. The mixture was dried over sodium sulfate and concentrated under vacuum. The crude product was offered as a brown solid which was used without further purification (15.4 g, 89%). An analytical sample was obtained by flash column chromatography (eluent: n-hexane:dichloromethane=3:1). ¹H NMR (400 MHz, DMSO-d₆) δ 7.98 (br, 2H). ¹³C{¹H} NMR (100 MHz, 353 K, DMSO-d₆) δ 152.7 (dd, J=262, 21.1 Hz) 100.7 (d, J=25.0 Hz). ¹⁹F NMR (376 MHz, DMSO-d₆) δ −122.06 (s, 1F), −125.52 (s, 2F). HRMS (Cl−) Calcd for C₆H₂F₂N₂O₂ (M⁻): 172.0084, Found: 172.0079.

### Step 2: Preparation of 5,6-difluoro-2,1,3-benzoxadiazole (3)

Compound 2 (12.0 g, 69.6 mmol) and triethyl phosphite (13.9 g, 83.5 mmol) were dissolved in tetrahydrofuran (150 mL). The mixture was refluxed overnight, cooled to room temperature and concentrated under vacuum. The residue was purified by flash column chromatography (eluent: n-hexane:dichloromethane=4:1). The product was obtained as a pale yellow solid (8.59 g, 79%). ¹H NMR (400 MHz, CDCl₃) δ 7.60 (t, J=7.6 Hz, 2H). ¹³C{¹H} NMR (100 MHz, CDCl₃) δ 154.6 (dd, J=266, 21.7 Hz), 146.1 (t, J=5.5 Hz), 101.2 (m). ¹⁹F NMR (376 MHz, CDCl₃) δ −121.03 (t, J=7.5 Hz, 2F). HRMS (Cl−) Calcd for C₆H₂F₂N₂O (M⁻): 156.0135, Found: 156.0134.

### Step 3: Preparation of 5,6-difluoro-4,7-bis (trimethylsilyl)-2,1,3-benzoxadiazole (4)

Compound 3 (19.7 g, 126 mmol) and trimethylsilyl chloride (48.0 mL, 378 mmol) were dissolved in dry tetrahydrofuran (200 mL) under nitrogen atmosphere. The solution was cooled to −78° C. and a lithium diisopropylamide solution (2 M, 139 mL, 278 mmol) was added dropwise. The mixture was kept at −78° C. for 2 h and then returned to room temperature overnight. The reaction was quenched by a saturated solution of ammonium chloride and extracted with diethyl ether for three times. The combined organic extract was washed with water and then brine. The mixture was dried over sodium sulfate and concentrated under vacuum. The crude product was purified by flash column chromatography (eluent: n-hexane). The product was obtained as a white solid (28.9 g, 76%). ¹H NMR (400 MHz, CDCl₃) δ 0.50 (s, 18H). ¹³C{¹H} NMR (100 MHz, CDCl₃) δ 157.5 (dd, J=262, 24.8 Hz), 149.4 (t, J=7.9 Hz), 113.3 (m), −0.3. ¹⁹F NMR (376 MHz, CDCl₃) δ −109.76 (s, 2F). HRMS (Cl−) Calcd for C₁₂H₁₈F₂N₂OSi₂ (M⁻): 300.0926, Found: 300.0929.

### Step 4: Preparation of 4,7-dibromo-5,6-difluoro-2,1,3-benzoxadiazole (5)

Compound 4 (16.9 g, 56.1 mmol) was dissolved in sulfuric acid (200 mL). N-bromosuccinimide (22.0 g, 124 mmol) was added in portions. The mixture was heated at 60° C. for 2 h, cooled to room temperature and carefully poured into ice. The precipitate was collected by filtration and purified by flash column chromatography (eluent: n-hexane:dichloromethane=6:1). The product was obtained as a white solid (11.0 g, 62%). ¹³C{¹H} NMR (100 MHz, CDCl₃) δ 152.5 (dd, J=267, 22.7 Hz), 146.0 (t, J=2.1 Hz), 94.0 (dd, J=17.6, 8.9 Hz). ¹⁹F NMR (376 MHz, CDCl₃) δ −114.14 (s, 2F). HRMS (Cl−) Calcd for C₆Br₂F₂N₂O (M⁺): 311.8345, Found: 311.8375.

### Step 5: Preparation of 5,6-difluoro-4,7-bis (4-(2-decyltetradecyl)-2-thienyl)-2,1,3-benzoxadiazole (6)

3-(2-Decyltetradecyl)thiophene-2-boronic acid pinacol ester (552 mg, 1.01 mmol), compound 5 (144 mg, 0.459 mmol), potassium carbonate (634 mg, 4.59 mmol), Pd(dba)₂ (26.4 mg, 0.0459 mmol) and 2-dicyclohexylphosphino-2′,6′-dimethoxybiphenyl (18.8 mg, 0.0459 mmol) were mixed under nitrogen atmosphere. Toluene (10 mL) and water (3 mL) were added. The mixture was refluxed overnight before cooled to room temperature. The mixture was diluted with diethyl ether and water. The organic layer was separated and washed with a saturated ammonium chloride aqueous solution, dried over sodium sulfate, and concentrated in vacuum. The residue was purified by flash column chromatography (eluent: n-hexane) to get the product as yellow solid (343 mg, 75%). ¹H NMR (400 MHz, CDCl₃) δ 8.10 (s, 2H), 7.18 (s, 2H), 2.63 (d, J=6.8 Hz, 4H) 1.75-1.63 (m, 2H), 1.46-1.14 (m, 80H), 0.93-0.82 (m, 12H). ¹³C{¹H} NMR (100 MHz, CDCl₃) δ 149.2 (dd, J=264, 21.9 Hz), 149.2 (t, J=4.1 Hz), 143.1, 133.4, 129.9, 125.7 (d, J=3.6 Hz), 107.6 (m), 39.1, 35.0, 33.5, 32.1, 30.2, 29.8, 29.8, 29.5, 26.8, 22.8, 14.3. ¹⁹F NMR (376 MHz, CDCl₃) δ −122.98 (s, 2F). HRMS (MALDI+) Calcd for C₆₂H₁₀₂F₂N₂OS₂ (M⁺): 992.7402, Found: 992.7439.

### Step 6: Preparation of 5,6-difluoro-4,7-bis(5-bromo-4-(2-decyltetradecyl)-2-thienyl)-2,1,3-benzoxadiazole (7)

A solution of compound 6 (343 mg, 0.345 mmol) in tetrahydrofuran (6 mL) was cooled to 0° C. in dark. N-bromosuccinimide (135 mg, 0.760 mmol) was added in one portion. The reaction mixture was warmed to room temperature, stirred overnight and concentrated in vacuum. The residue was purified by flash column chromatography (eluent: n-hexane) to get the product as orange solid (230 mg, 58%). ¹H NMR (400 MHz, CDCl₃) δ 7.96 (s, 2H), 2.58 (d, J=7.2 Hz, 4H) 1.74 (br, 2H), 1.45-1.12 (m, 80H), 0.92-0.80 (m, 12H). ¹³C{¹H} NMR (100 MHz, CDCl₃) δ 149.1 (dd, J=264, 21.7 Hz), 144.8 (t, J=4.1 Hz), 142.6, 133.1, 129.7, 115.9 (t, J=5.0 Hz), 107.2 (dd, J=11.9, 4.1 Hz), 38.7, 34.3, 33.5, 32.1, 30.1, 29.9, 29.8, 29.8, 29.5, 26.7, 22.8, 14.3. ¹⁹F NMR (376 MHz, CDCl₃) δ −122.93 (s, 2F). HRMS (MALDI+) Calcd for C₆₂H₁₀₀Br₂F₂N₂OS₂ (M⁺): 1150.5591, Found: 1150.5554.

### Example 2—Polymer Synthesis

In an embodiment, the present subject matter is directed to synthesis of polymer PffBX4T-2DT according to the following synthetic route:

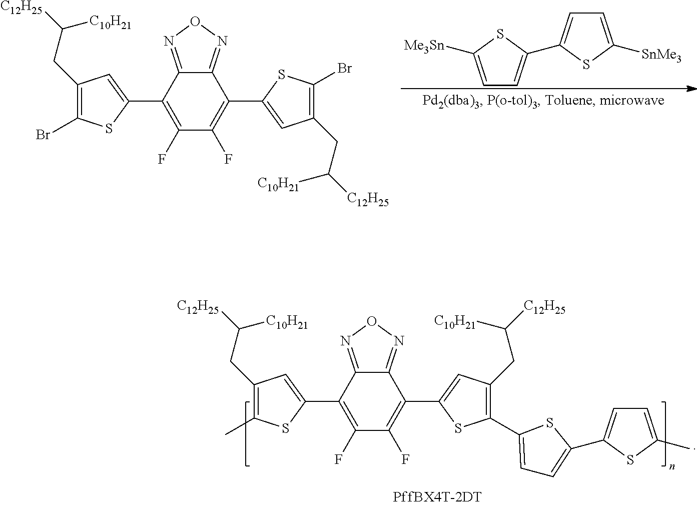

A mixture of monomer 7 (66.4 mg, 0.0578 mmol), 5,5′-bis(trimethylstannyl)-2,2′-bithiophene (Sunatech IN1207, 28.4 mg, 0.0578 mmol), Pd₂(dba)₃ (0.5 mg, 0.0005 mmol) and P(o-tol)₃ (1.0 mg, 0.0033 mmol) was placed in a microwave tube. Toluene (0.2 mL) was added in a glove box which is filled with nitrogen. The tube was sealed and heated to 140° C. for 1 h in a microwave reactor. The obtained deep green gel was diluted with 20 mL hot 1,2-dichlorobenzene and the solution was precipitated into methanol. The solid was collected by filtration, and loaded into a thimble in a Soxhlet extractor. The crude polymer was extracted successively with acetone, chloroform and toluene. The toluene solution was concentrated by evaporation, re-dissolved in hot chlorobenzene and precipitated into methanol. The solid was collected by filtration and dried in vacuo to get the polymer as black solid (67 mg, 78%). ¹H NMR (400 MHz, 393 K, C₂D₂Cl₄). δ 8.17 (s, 2H), 7.24 (br, 4H), 2.94 (d, J=7.2 Hz, 4H), 1.99-1.85 (m, 2H), 1.54-1.25 (m, 80H), 0.99-0.89 (m, 12H). Elem. Anal. Calcd for C₇₀H₁₀₄F₂N₂OS₄: C, 72.74; H, 9.07; N, 2.42. Found: C, 72.66; H, 9.17; N, 2.42. HT-GPC: Mₙ=159 kDa, M_{w}=332 kDa, PDI=2.09.

### Example 3—Characterization of Polymers

Optical Properties

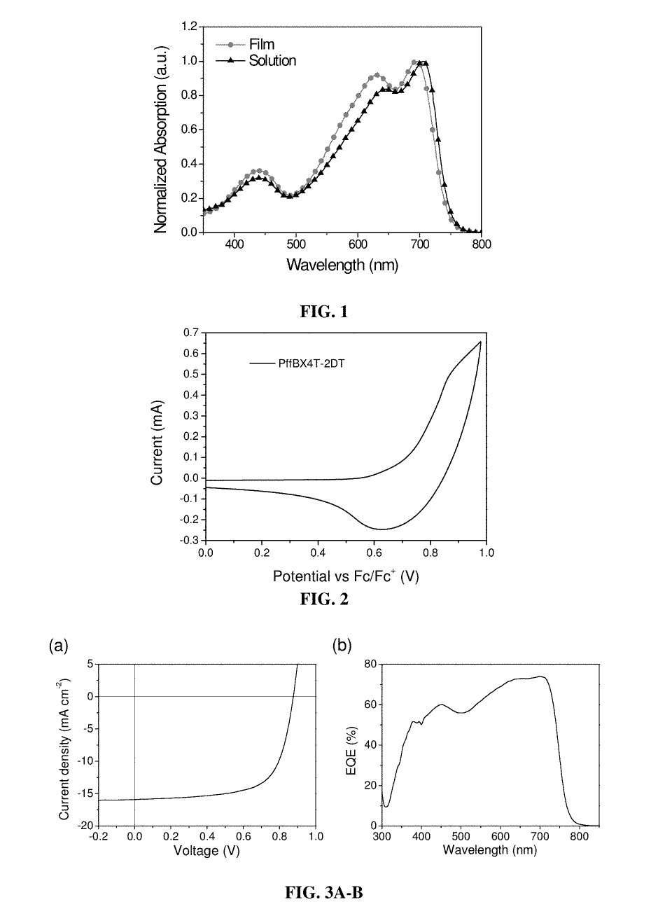

Film UV-Vis absorption spectra of polymers from Example 2 were acquired on a Perkin Elmer Lambda 20 UV/VIS Spectrophotometer. All film samples were spin-cast on ITO/ZnO substrates. Solution UV-Vis absorption spectra at elevated temperatures were collected on a Perkin Elmer Lambda 950 UV/VIS/NIR Spectrophotometer. The temperature of the cuvette was controlled with a Perkin Elmer PTP 6+6 Peltier System, which is supplied by a Perkin Elmer PCB 1500 Water Peltier System. Before each measurement, the system was held for at least 10 min at the target temperature to reach thermal equilibrium. A cuvette with a stopper (Sigma Z600628) was used to avoid volatilization during the measurement. The onset of the absorption is used to estimate the polymer bandgap. The optical absorption spectrum is shown in FIG. 1.

Electronic Properties

Cyclic voltammetry was carried out on a CHI760E electrochemical workstation with three electrodes configuration, using Ag/AgCl as the reference electrode, a Pt plate as the counter electrode, and a glassy carbon as the working electrode. Polymers were drop-cast onto the electrode from DCB solutions to form thin films. 0.1 mol L⁻¹ tetrabutylammonium hexafluorophosphate in anhydrous acetonitrile was used as the supporting electrolyte. Potentials were referenced to the ferrocenium/ferrocene couple by using ferrocene as external standards in acetonitrile solutions. The scan rate is 0.1 V s⁻¹ (shown in FIG. 2).

### Example 4—Device Fabrication

Photovoltaic Cell Fabrication and Measurements

Pre-patterned ITO-coated glass with a sheet resistance of −15 S2 per square was used as the substrate. It was cleaned by sequential ultrasonications in soap deionized water, deionized water, acetone and isopropanol for 15 min at each step. The washed substrates were further treated with a UV—O₃ cleaner (Novascan, PSD Series digital UV ozone system) for 30 min. A topcoat layer of ZnO (The diethylzinc solution 15 wt % in toluene, diluted with tetrahydrofuran) was spin-coated onto the ITO substrate at a spinning rate of 5000 rpm for 30 s and then baked in air at 150° C. for 20 min.

Active layer solutions (polymer:fullerene weight ratio 1:1.2) were prepared in DCB with 1% DIO. The polymer concentration is 8 mg ml⁻¹. To completely dissolve the polymer, the active layer solution was stirred on a hot plate at 100° C. for at least 1 h. Before spin coating, both the polymer solution and ITO substrate were preheated on a hot plate at −110° C. Active layers were spin coated from the warm polymer solution onto the preheated substrate in a N₂ glovebox at −700 rpm. The active layers were then treated with vacuum to remove the high boiling point additives. The blend films were annealed at 80° C. for 5 min before being transferred to the vacuum chamber of a thermal evaporator inside the same glovebox. At a vacuum level of 3×10⁻⁶ Torr, a thin layer (20 nm) of V₂O₅ was deposited as the anode interlayer, followed by deposition of 100 nm of Al as the top electrode. All cells were encapsulated using epoxy inside the glovebox.

Device J-V characteristics was measured under air mass 1.5 global (100 mW cm²) using a Newport Class A solar simulator (94021A, a Xenon lamp with an AM1.5G filter). A standard crystalline Si solar cell with a KG5 filter was purchased from PV Measurements and calibrated by Newport Corporation. The light intensity was calibrated using the standard Si diode to bring spectral mismatch to unity. J-V characteristics were recorded using a Keithley 236 or 2400 source meter unit. Typical cells have devices area of −5.9 mm², which is defined by a metal mask with an aperture aligned with the device area. EQEs were characterized using a Newport EQE system equipped with a standard Si diode. Monochromatic light was generated from a Newport 300 W lamp source. The V_{OC}, J_{SC}, FF and PCE of OPV devices in the present teaching are summarized in the following table. The J-V and EQE curves are shown in FIG. 3.

Table 1 contains data for the solar cell performance of PffBX4T-2DT:PC₇₁BM at different thicknesses. The averages and standard derivations were calculated from at least 5 devices.

TABLE 1
Solar cell performance of PffBX4T-2DT:PC₇₁ BM at different thicknesses

| | | J_{SC} | | PCE |
|---|---|---|---|---|
| | | [mA | | (best PCE) |
| Thickness | V_{OC} [V] | cm⁻²] | FF | [%] |
| 110 ± 10 nm | 0.878 ± 0.004 | 13.6 ± 0.2 | 0.72 ± 0.01 | 8.6 ± 0.2 (8.9) |
| 250 ± 10 nm | 0.875 ± 0.003 | 15.8 ± 0.1 | 0.66 ± 0.02 | 9.1 ± 0.3 (9.4) |
| 300 ± 10 nm | 0.867 ± 0.006 | 15.9 ± 0.1 | 0.62 ± 0.01 | 8.5 ± 0.2 (8.7) |

### Example 5—Synthesis of Monomers

In an embodiment, the present subject matter is directed to synthesis of monomers according to the following synthetic route.

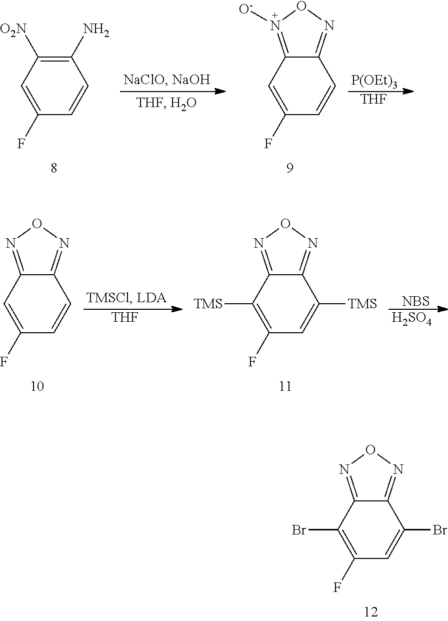

### Step 1: Preparation of 5-fluoro-2,1,3-benzoxadiazole 1-oxide (9)

A mixture of compound 8 (1.561 g, 10.0 mmol), sodium hydroxide (120 mg, 3.0 mmol) and tetrahydrofuran (20 mL) was cooled to 0° C. Sodium hypochlorite solution (13% available chlorine, 9 mL) was added dropwise. Upon addition, the mixture was stirred at 0° C. for another 2 h. The mixture was diluted with water and extracted with dichloromethane for three times. The organic extract was combined and washed successively with water and a saturated solution of ammonium chloride. The mixture was dried over sodium sulfate and concentrated under vacuum. The crude product was offered as a brown solid which was used without further purification.

### Step 2: Preparation of 5-fluoro-2,1,3-benzoxadiazole (10)

Compound 9 (obtained from previous step) and triethyl phosphite (1.99 g, 12.0 mmol) were dissolved in tetrahydrofuran (23 mL). The mixture was refluxed overnight, cooled to room temperature and concentrated under vacuum. The residue was purified by flash column chromatography (eluent: n-hexane:dichloromethane=3:1), and a yellow oil was obtained as the product (700 mg, 51% for two steps).

### Step 3: Preparation of 5-fluoro-4,7-bis(trimethylsilyl)-2,1,3-benzoxadiazole (11)

Compound 10 (700 mg, 5.07 mmol) and trimethylsilyl chloride (1.9 mL, 15.2 mmol) were dissolved in dry tetrahydrofuran (10 mL) under nitrogen atmosphere. The solution was cooled to −78° C. and a lithium diisopropylamide solution (2 M, 5.6 mL, 11.2 mmol) was added dropwise. The mixture was kept at −78° C. for 2 h and then returned to room temperature overnight. The reaction was quenched by a saturated solution of ammonium chloride and extracted with diethyl ether for three times. The combined organic extract was washed with water and then brine. The mixture was dried over sodium sulfate and concentrated under vacuum. The crude product was purified by flash column chromatography (eluent: n-hexane). The product was obtained as a white solid (686 mg, 48%).

### Step 4: Preparation of 4,7-dibromo-5,6-difluoro-2,1,3-benzoxadiazole (12)

Compound 11 (686 mg, 2.43 mmol) was dissolved in sulfuric acid (10 mL). N-bromosuccinimide (951 mg, 5.34 mmol) was added in portions. The mixture was heated at 60° C. for 2 h, cooled to room temperature and carefully poured into ice. The precipitate was collected by filtration and purified by flash column chromatography (eluent: n-hexane:dichloromethane=6:1). The product was obtained as a white solid (384 mg, 54%).

With the information contained herein, various departures from precise descriptions of the present subject matter will be readily apparent to those skilled in the art to which the present subject matter pertains, without departing from the spirit and the scope of the below claims. The present subject matter is not considered limited in scope to the procedures, properties, or components defined, since the preferred embodiments and other descriptions are intended only to be illustrative of particular aspects of the presently provided subject matter. Indeed, various modifications of the described modes for carrying out the present subject matter which are obvious to those skilled in chemistry, biochemistry, or related fields are intended to be within the scope of the following claims.

We claim:

1. Polymer comprising one or more repeating units of formula I: 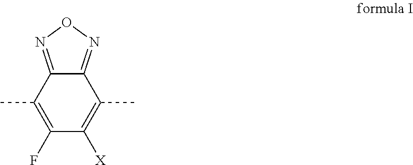
wherein X is H or F.

2. Polymer according to claim 1, wherein the units of formula I are selected from formulae II and

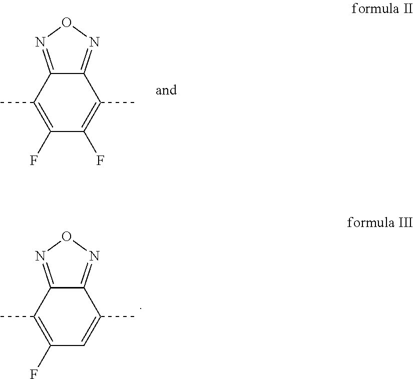

3. Polymer according to claim 1, characterized in that it comprises one or more repeating units of formula IV: 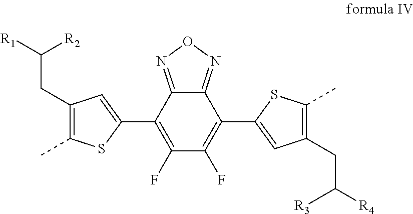
wherein R₁, R₂, R₃ and R₄, at each occurrence, independently can be a C₁₋₄₀ alkyl group.

4. Polymer according to claim 1, characterized in that it comprises one or more repeating units of formula V: 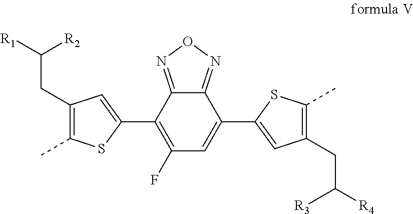
wherein R₁, R₂, R₃ and R₄, at each occurrence, independently can be a C₁₋₄₀ alkyl group.

5. Polymer according to claim 1, characterized in that it comprises one or more repeating units of formula VI:

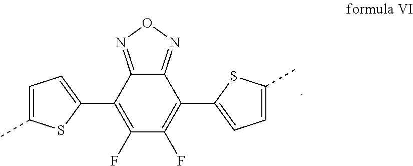

6. Polymer according to claim 1, characterized in that it comprises one or more repeating units of formula VII:

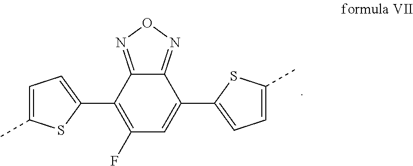

7. Polymer according to claim 1, characterized in that it comprises one or more repeating units of formula X: 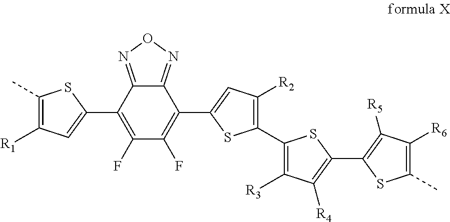
wherein R₁, R₂, R₃, R₄, R₅ and R₆, at each occurrence, independently can be a C₁₋₄₀ alkyl group.

8. Polymer according to claim 1, characterized in that it comprises one or more repeating units of formula XI: 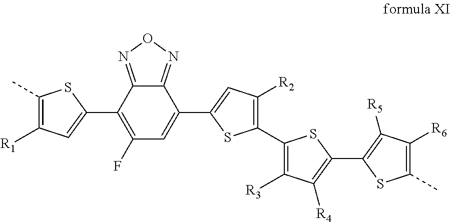
wherein R₁, R₂, R₃, R₄, R₅ and R₆, at each occurrence, independently can be a C₁₋₄₀ alkyl group.

9. Polymer according to claim 1, characterized in that it comprises one or more repeating units of formula XII: 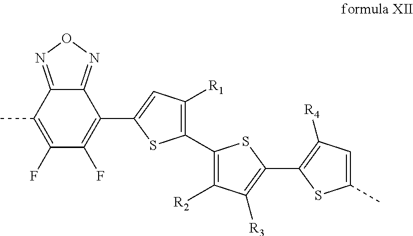
wherein R₁, R₂, R₃ and R₄, at each occurrence, independently can be a C₁₋₄₀ alkyl group.

10. Polymer according to claim 1, characterized in that it comprises one or more repeating units of formula XIII: 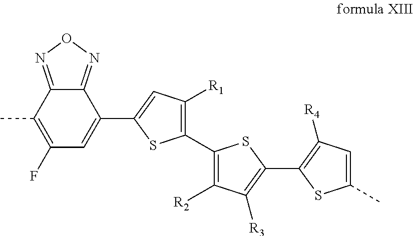
wherein R₁, R₂, R₃ and R₄, at each occurrence, independently can be a C₁₋₄₀ alkyl group.

11. Polymer according to claim 1, characterized in that it comprises one or more repeating units selected from: 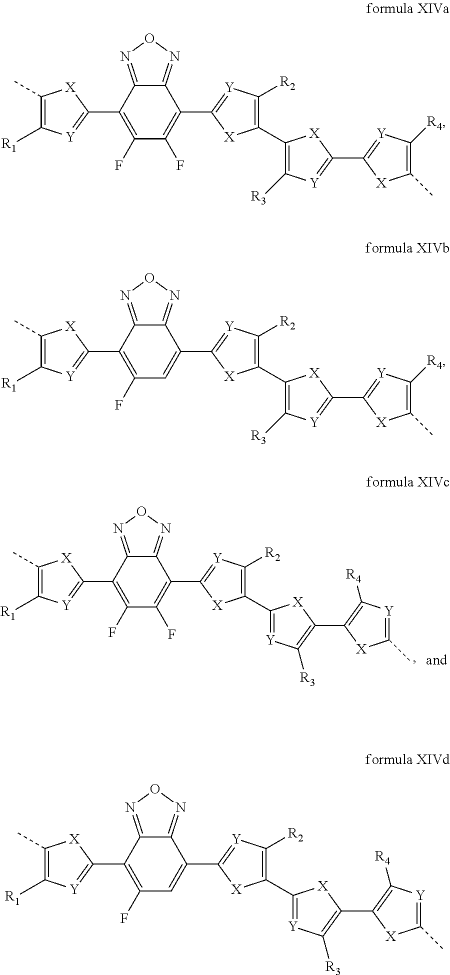
wherein
R₁, R₂, R₃ and R₄, at each occurrence, independently can be a C1-40 alkyl group;
each X is independently selected from the group consisting of O, S, Se and Te; and
each Y is independently selected from the group consisting of N, C—H, and C—R5, wherein R5 is selected from the group consisting of C1-40 straight-chain and branched alkyl groups.

12. Polymer according to claim 1, characterized in that it comprises one or more repeating units selected from: 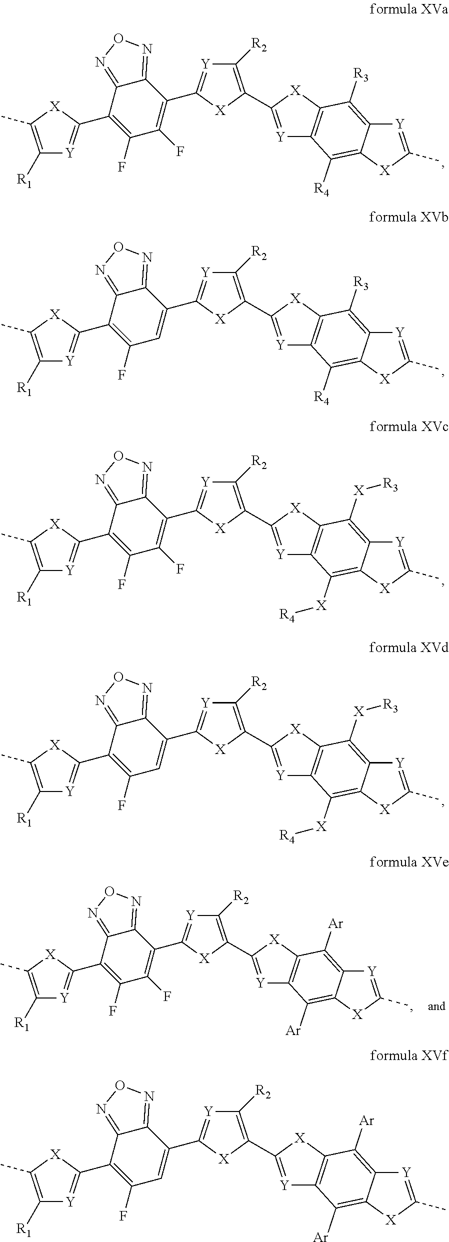
wherein
R₁, R₂, R₃ and R₄, at each occurrence, independently can be a C1-40 alkyl group;
each X is independently selected from the group consisting of O, S, Se and Te;
each Y is independently selected from the group consisting of N, C—H, and C—R5, wherein R5 is selected from the group consisting of C1-40 straight-chain and branched alkyl groups; and
each Ar is independently selected from the group consisting of unsubstituted or substituted monocyclic, bicyclic, and polycyclic arylene, and monocyclic, bicyclic, and polycyclic heteroarylene, wherein each Ar may contain one to five of said arylene or heteroarylene each of which may be fused or linked.

13. Polymer according to claim 1, characterized in that it comprises one or more repeating units selected from: 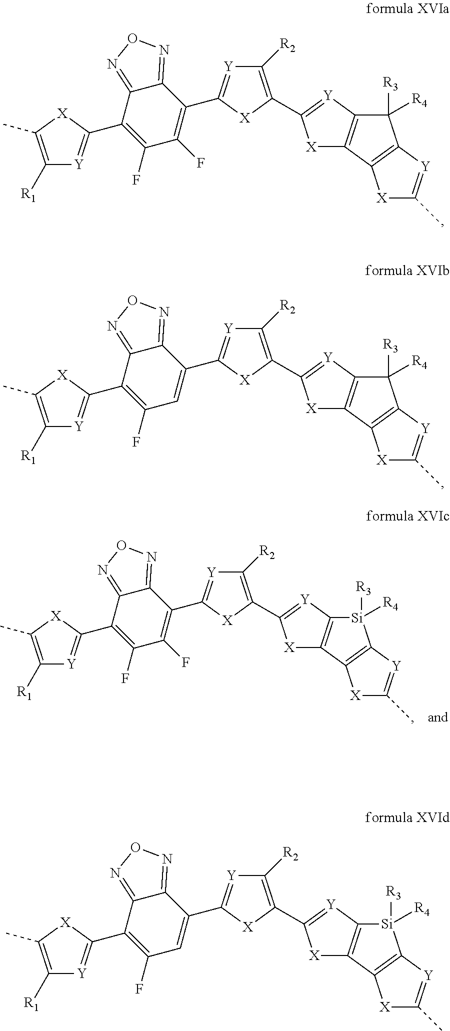
wherein
R₁, R₂, R₃ and R₄, at each occurrence, independently can be a C1-40 alkyl group;
each X is independently selected from the group consisting of O, S, Se and Te; and
each Y is independently selected from the group consisting of N, C—H, and C—R5, wherein R5 is selected from the group consisting of C1-40 straight-chain and branched alkyl groups.

14. Polymer according to claim 1, characterized in that it comprises one or more repeating units selected from: 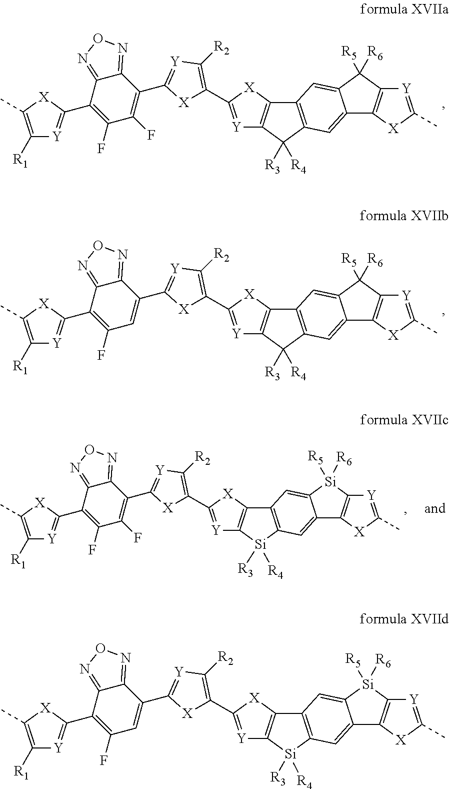
wherein
R₁, R₂, R₃, R₄, R₅ and R₆, at each occurrence, independently can be a C1-40 alkyl group;
each X is independently selected from the group consisting of O, S, Se and Te; and
each Y is independently selected from the group consisting of N, C—H, and C—R5, wherein R5 is selected from the group consisting of C1-40 straight-chain and branched alkyl groups.

15. A process of preparing a polymer or organic compound comprising polymerizing an intermediate with formula VIII: 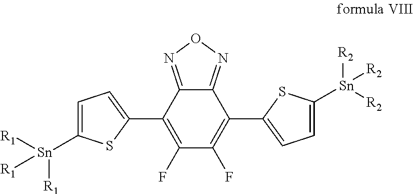
wherein R₁ and R₂, at each occurrence, independently can be a C₁₋₁₀ alkyl group.

16. A process of preparing a polymer or organic compound comprising polymerizing an intermediate with formula IX: 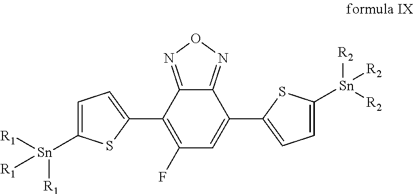
wherein R₁ and R₂, at each occurrence, independently can be a C₁₋₁₀ alkyl group.

17. A formulation comprising:
the polymer of claim 1, and
a fullerene, a second polymer, or a small molecule.

18. An organic electronic (OE) device comprising a coating or printing ink containing the formulation of claim 17.

19. The OE device of claim 18, characterized in that the OE device is an organic field effect transistor (OFET) device.

20. The OE device of claim 18, characterized in that the OE device is an organic photovoltaic (OPV) device.

21. A coating or printing ink comprising the formulation of claim 17.

22. The coating or printing ink of claim 21, wherein the coating or printing ink is for preparing OE devices and rigid or flexible OPV cells and devices.

23. An organic electronic (OE) device prepared from the formulation of claim 17.

24. A synthesis of monomers comprising one or more of the following steps:
reacting 4,5-difluoro-2-nitroaniline (Compound 1) with a base, for example sodium hydroxide or potassium hydroxide, in an organic solvent mixture containing solvents, for example tetrahydrofuran or 1,4-dioxane, via a ring-closure reaction, to produce 5,6-difluoro-2,1,3-benzoxadiazole 1-oxide (Compound 2);
reacting Compound 2 with a reductant, for example triethyl phosphite or triphenyl phosphite, in an organic solvent mixture containing solvents, for example tetrahydrofuran or toluene, via a reduction reaction, to obtain 5,6-difluoro-2,1,3-benzoxadiazole (Compound 3);
reacting Compound 3 with a Lewis acid, for example trimethylsilyl chloride, and a base, for example lithium diisopropylamide, in an organic solvent mixture containing solvents, for example tetrahydrofuran, via a substitution reaction, to obtain 5,6-difluoro-4,7-bis(trimethylsilyl)-2,1,3-benzoxadiazole (Compound 4); and
reacting Compound 4 with a bromination reagent, for example N-bromosuccinimide, in an organic solvent mixture containing solvents, for example sulfuric acid, via a substitution reaction, to obtain 4,7-dibromo-5,6-difluoro-2,1,3-benzoxadiazole (Compound 5).

25. A monomer prepared according to the synthesis of claim 24.

26. A synthesis of monomers comprising one or more of the following steps:
reacting 4-fluoro-2-nitroaniline (Compound 8) with a base, for example sodium hydroxide or potassium hydroxide, in an organic solvent mixture containing solvents, for example tetrahydrofuran or 1,4-dioxane, via a ring-closure reaction to obtain 5-fluoro-2,1,3-benzoxadiazole 1-oxide (Compound 9);
reacting Compound 9 with a reductant, for example triethyl phosphite or triphenyl phosphite, in an organic solvent mixture containing solvents, for example tetrahydrofuran or toluene, via a reduction reaction, to obtain 5-fluoro-2,1,3-benzoxadiazole (Compound 10);
reacting Compound 10 with a Lewis acid, for example trimethylsilyl chloride, and a base, for example lithium diisopropylamide, in an organic solvent mixture containing solvents, for example tetrahydrofuran, via a substitution reaction, to obtain 5-fluoro-4,7-bis(trimethylsilyl)-2,1,3-benzoxadiazole (Compound 11); and
reacting Compound 11 with a bromination reagent, for example N-bromosuccinimide, in an organic solvent mixture containing solvents, for example sulfuric acid, via a substitution reaction to obtain 4,7-dibromo-5,6-difluoro-2,1,3-benzoxadiazole (Compound 12).

27. A monomer prepared according to the synthesis of claim 26.
